(19) United States
(12) Patent Application Publication
(10) Pub. No.: US 2006/0107469 A1
(43) Pub. Date: May 25, 2006

(54) Formulations designed to be applied on keratinous material and to be rinsed

(76) Inventors: Olivier Anthony, Meriel (FR); Cedric Geffroy, Poitiers (FR)

Correspondence Address: Jean Louis Seugnet;Intellectual Property Department, Rhodia Inc CN7500, 259 Prospect Plains Road, Cranbury, NJ, 08512-7500, US

(21) Appl. No.: 10/527,071

(22) PCT Filed: Aug. 26, 2003
(86) PCT No.: PCT/FR03/02579

§ 371 Date: Jan. 11, 2006

Related U.S. Application Data
(60) Provisional application No. 60/409,352, filed on Sep. 9, 2002.

(51) Int. Cl. A61K 8/00

(52) U.S. Cl. 8/405

(57) ABSTRACT

The invention concerns a formulation designed to be applied on keratinous material such as the skin, the hair, and to be rinsed with an aqueous rinsing medium, in the form of a stable dispersion for a pH ranging between 3 and 5.5, and comprising at least one active substance, a vector agent consisting of at least one organic polymer capable of bringing said active substance to the surface of the keratinous material during the rinsing process, and optionally at least one salt soluble in the formulation; the active substance, whether or not in liquid form, has in the medium of the formulation, a global cationic or zero charge; it is insoluble in the formulation medium and stabilized by means of a cationic surfactant and it remains insoluble or tends to swell in the rinsing medium; the invention also concerns methods for treating keratinous material using said formulation, as well as the use of a vector agent consisting of at least an organic polymer in the formulation as agent capable of bringing the active matter to the surface of the keratinous matter during the rinsing process

The present invention relates to a formulation intended to be applied to keratinous material, such as the skin or the hair, and then to be rinsed. It similarly relates to methods for treating keratinous material and to the use of a polymer as an agent for depositing an active material at the surface of keratinous material.

It has been found that the addition, to a formulation intended to be rinsed and comprising an active material that is insoluble in this formulation, whether the latter is either in the form of a liquid or in the form of solid particles, which may or may not be polymeric, dispersed in said formulation, of a small amount of a suitably chosen soluble carrier agent makes it possible to improve the deposition of the active material at the surface of keratinous material, and thus to provide the treated material with notable benefits, in particular a protection, a volumizing effect, a fixing effect, easy styling, etc.

A first subject of the invention therefore consists of a formulation intended to be applied to keratinous material and to be rinsed with an aqueous rinsing medium in the form of a stable dispersion, the pH of which is between 3 and 5.5, and comprising at least one active material, a carrier agent consisting of at least one organic polymer capable of bringing said active material to the surface of the keratinous material during the rinsing process and, optionally, at least one salt that is soluble in the formulation; the nature of the active material and of the carrier agent being such that:

the active material:
may or may not be in a liquid form,
has, in the medium of the formulation, an overall cationic or zero charge,
is insoluble in the medium of the formulation,
is stabilized in the medium of the formulation by means of a cationic and/or nonionic surfactant,
remains insoluble in the rinsing medium or is capable of swelling in the rinsing medium;

the carrier agent:
is soluble or dispersible in the medium of the formulation and in the rinsing medium,
has, in the medium of the formulation, an overall ionic charge that is zero or cationic,
is capable of developing, at the pH of the rinsing process in the rinsing medium, a sufficient number of anionic charges to destabilize the active material in the rinsing medium.

A second subject of the invention consists of a method of treating keratinous material by bringing said material into contact with the abovementioned formulation and then rinsing it with an aqueous rinsing medium.

The invention similarly relates to a method intended to improve the volumizing properties and/or the properties that help styling and/or the properties consisting of a fixing effect for keratinous fibers, consisting in bringing said fibers into contact with the formulation according to the invention, and then in rinsing said fibers with an aqueous rinsing medium.

It similarly relates to a method for improving the depositing of an active material onto keratinous material, during which a formulation is applied to said material and then a rinsing process is carried out with an aqueous rinsing medium;

said formulation comprising at least one active material and, optionally, at least one salt that is soluble in the formulation, and being in the form of a stable dispersion, the pH of which is between 3 and 5.5,

the active material containing, optionally in encapsulated, dispersed or solubilized form, at least one hydrophobic organic active compound that is different from the active material; said active material, which may or may not be in a liquid form, having, in the medium of the formulation, an overall cationic or zero charge, being insoluble in the medium of the formulation, being stabilized in the medium of the formulation by means of a cationic and/or nonionic surfactant, and remaining insoluble in the rinsing medium or being capable of swelling in the rinsing medium;

by addition of at least one carrier agent consisting of at least one organic polymer that is soluble or dispersible in the medium of the formulation and in the rinsing medium, having, in the medium of the formulation, an overall ionic charge that is zero or cationic and being capable of developing, at the pH of the rinsing process in the rinsing medium, a sufficient number of anionic charges to destabilize the active material in the rinsing medium.

The invention similarly relates to the use, in a formulation intended to be applied to keratinous material and to be rinsed with an aqueous or aqueous-alcoholic rinsing medium, said formulation being in the form of a stable dispersion, the pH of which is between 3 and 5.5, and comprising:

at least one active material, which may or may not be in a liquid form, that has, in the medium of the formulation, an overall cationic or zero charge, that is insoluble in the medium of the formulation, that is stabilized in the medium of the formulation by means of a cationic and/or nonionic surfactant, and that remains insoluble in the rinsing medium or is capable of swelling in the rinsing medium;

optionally, at least one salt that is soluble in the formulation;

of at least one carrier agent consisting of at least one organic polymer that is soluble or dispersible in the medium of the formulation and in the rinsing medium, that has, in the medium of the formulation, an overall ionic charge that is zero or cationic and that is capable of developing, at the pH of the rinsing process in the rinsing medium, a sufficient number of anionic charges to destabilize the active material in the rinsing medium,

as an agent capable of bringing said active material to the surface of the keratinous fibers during the rinsing process.

For that which follows, it is considered that a dispersion of particles of a liquid or of a solid in a medium is considered to be stable if no sedimentation, phase-separation or change in turbidity is observed over time. This dispersion destabilizes when the particles aggregate with one another or coalesce.

According to the invention, the active material is considered to be destabilized in the rinsing medium comprising the carrier agent when the turbidity of said medium is at least 5 times greater than the turbidity that the same medium would have in the absence of carrier agent.

As was previously indicated, the formulation according to the invention is in the form of a stable dispersion, the pH of which is between 3 and 5.5, more particularly between 4.5 and 5.5.

The medium of the formulation is more particularly an aqueous or aqueous-alcoholic formulation.

Among the alcohols that may be present, mention may be made of ethanol, isopropanol, propylene glycol, butoxyethanol, etc.

These alcohols can represent up to 70% of the volume of the medium of the formulation.

Preferably, the medium of the formulation is water.

It should be noted that, according to the elements present in the formulation, said formulation can be brought to the desired pH of between 3 and 5.5, more particularly of between 4.5 and 5.5, by adding an acid, such as hydrochloric acid, citric acid, phosphoric acid, benzoic acid, etc.

The formulation comprises, moreover, at least one active agent, a carrier agent and, optionally, at least one salt that is soluble in the formulation.

The active material that goes to make up the composition of the formulation may or may not be in a liquid form.

The active material is, moreover, insoluble in the medium of the formulation and remains insoluble in the rinsing medium or is capable of swelling in this same medium.

Thus, the active material consists more particularly of dispersed solid or liquid particles, or even of a combination of both.

In addition, the active material is stabilized in the medium of the formulation by means of a cationic surfactant, which will subsequently be described.

According to another characteristic, the active material has, in the medium of the formulation, an overall cationic or zero charge.

In accordance with a first variant of the invention, the active material is a solid organic polymer in the form of particles dispersed in the medium of the formulation.

It should be noted that the term “polymer” is here used to denote both a homopolymer and a copolymer.

Among the copolymers that may constitute the active material, mention may be made of:

a) nonionic polymers derived from at least one nonionic hydrophobic monomer,

b) polymers derived from at least one nonionic hydrophobic monomer and from at least one monomer that is cationic or potentially cationic in the medium of the formulation and, optionally, from at least one monomer that is neutral in the medium of the formulation and potentially anionic in the rinsing medium,

c) polymers derived from at least one nonionic hydrophobic monomer and from at least one monomer that is neutral in the medium of the formulation and potentially anionic in the rinsing medium.

The monomer composition from which said polymer derives can also contain:
at least one uncharged or non-ionizable hydrophilic monomer, preferably in an amount that does not exceed 50% of the total mass of monomers,
and/or at least one zwitterionic monomer, preferably in an amount that does not exceed 30% of the total mass of monomers,
and/or at least one crosslinking monomer, preferably in an amount that does not exceed 10% of the total mass of monomers.

When the polymers b) above are involved, the monomer composition from which said polymers derive can optionally also contain a small amount of anionic monomer, the first pKa of which is less than 3, the polymer b) having to have, however, in the medium of the formulation, an overall cationic charge.

When ionic or ionizable polymers are involved, the choice and the relative amounts of monomers from which said polymers derive are such that the active material is insoluble in the medium of the formulation, has, in the medium of the formulation, an overall cationic or zero charge, remains insoluble in the rinsing medium or is not capable of swelling by more than 8 times, preferably not by more than 4 times, its volume in the rinsing medium.

Thus, when a monomer that is neutral in the medium of the formulation and potentially anionic in the rinsing medium is present in the monomer composition, said monomer preferably does not represent more than 50% of the total mass of monomers, so that the polymer obtained does not swell by more than 8 times, preferably not by more than 4 times, its volume in the rinsing medium.

When an anionic monomer (the first pKa of which is less than 3) is present in the monomer composition, said monomer preferably does not represent more than 20%, more particularly not more than 10%, of the total mass of monomers, so that said polymer has, in the medium of the formulation, an overall cationic charge.

When a zwitterionic monomer is present in the monomer composition, said monomer preferably does not represent more than 30%, preferably not more than 20%, of the total mass of monomers, so that said polymer has, in the medium of the formulation, an overall cationic charge.

According to this variant of the invention, the active material is in the form of solid particles of polymer. The particles may have a mean diameter of between 10 nm and 10 μm, preferably between 10 nm and 1 μm, and more preferably between 10 nm and 500 nm. The diameter of the particles can be determined in a well-known manner by light diffraction or by transmission electron microscopy.

Preferably, the active material is a polymer that derives from monomers that are α-β monoethylenically unsaturated or diethylenically unsaturated in the case of the crosslinking monomers.

By way of examples of hydrophobic nonionic monomers, mention may be made of:
vinylaromatic monomers such as styrene, vinyltoluene, etc.,
alkyl esters of α-β monoethylenically unsaturated acids, such as methyl, ethyl, etc. acrylates and methacrylates,
vinyl esters or allyl esters of saturated carboxylic acids, such as vinyl or allyl acetates, propionates or versatates,
α-β monoethylenically unsaturated nitrites such as acrylonitrile, etc.,
α-olefins such as ethylene, etc.

By way of examples of hydrophilic monomers that are cationic in the medium of the formulation, mention may be made of:
acryloyl- or acryloyloxyammonium monomers such as trimethylammonium propyl methacrylate chloride, trimethylammonium ethylacrylamide or -methacrylamide chloride or bromide, trimethylammonium butylacrylamide or -methacrylamide methyl sulfate, trimethylammonium propylmethacrylamide methyl sulfate (MES), (3-methacrylamidoproyl)trimethylammonium chloride (MAPTAC), (3-acrylamidopropyl)trimethylammonium chloride (APTAC), methacryloyloxyethyltrimethylammonium chloride or methyl sulfate, and acryloyloxyethyltrimethylammonium chloride;
1-ethyl-2-vinylpyridinium or 1-ethyl-4-vinylpyridinium bromide, chloride or methyl sulfate;
N,N-dialkyldiallylamine monomers such as N,N-dimethyldiallylammonium chloride (DADMAC);
polyquaternary monomers such as dimethylaminopropylmethacrylamide chloride and N-(3-chloro-2-hydroxypropyl)trimethylammonium (DIQUAT), etc.,
carboxybetaine monomers.

By way of examples of hydrophilic monomers that are potentially cationic in the medium of the formulation, mention may be made of:
N,N-(dialkylamino-ω-alkyl)amides of α-β monoethylenically unsaturated carboxylic acids such as N,N-dimethylaminoethylacrylamide or -methacrylamide, 2-(N,N-dimethylamino)ethylacrylamide or -methacrylamide, 3-(N,N-dimethylamino)propylacrylamide or -methacrylamide, and 4-(N,N-dimethylamino)butylacryl-amide or -methacrylamide,
α-β monoethylenically unsaturated amino esters such as 2-(dimethylamino)ethyl methacrylate (DMAM), 3-(dimethylamino)propyl methacrylate, 2-(tert-butylamino)ethyl methacrylate, 2-(dipentylamino)ethyl methacrylate, and 2-(diethylamino)ethyl methacrylate,
monomers which are precursors of amine functions, such as N-vinylformamide, N-vinylacetamide, etc., which give rise to primary amine functions by simple acid or base hydrolysis.

By way of examples of hydrophilic monomers that are neutral in the medium of the formulation and potentially anionic in the rinsing medium, mention may be made of:
monomers having at least one carboxylic function, for instance α-β ethylenically unsaturated carboxylic acids or the corresponding anhydrides, such as acrylic, methacrylic or maleic acids or anhydrides, fumaric acid, itaconic acid, N-methacroylalanine, N-acryloylglycine, and their water-soluble salts,
monomers which are precursors of carboxylate functions, such as tert-butyl acrylate, which, after polymerization, give rise to carboxylic functions by hydrolysis.

By way of examples of hydrophilic monomers that are anionic in the medium of the formulation (the first pKa of which is less than 3), mention may be made of:
monomers having at least one sulfate or sulfonate function, such as 2-sulfoxyethyl methacrylate, vinylbenzene sulfonic acid, allyl sulfonic acid, 2-acrylamido-2-methylpropane sulfonic acid, sulfoethyl acrylate or methacrylate, sulfopropyl acrylate or methacrylate, and their water-soluble salts,
monomers having at least one phosphonate or phosphate function, such as vinylphosphonic acid, etc., the esters of ethylenically unsaturated phosphates, such as the phosphates derived from hydroxyethyl methacrylate (Empicryl 6835 from Rhodia) and those derived from polyoxyalkylene methacrylates, and their water-soluble salts.

By way of examples of hydrophilic monomers that are uncharged or non-ionizable, mention may be made of:
hydroxyalkyl esters of α-β ethylenically unsaturated acids, such as hydroxyethyl and hydroxypropyl acrylate and methacrylate, glyceryl monomethacrylate, etc.,
α-β ethylenically unsaturated amides such as acrylamide, N,N-dimethylmethacrylamide, N-methylolacrylamide, etc.,
α-β ethylenically unsaturated monomers bearing a water-soluble polyoxyalkylene segment of the polyethylene oxide type, such as polyethylene oxide α-methacrylates (Bisomer S20W, S10W, etc., from Laporte) or α,ω-dimethacrylates, Sipomer BEM from Rhodia (ω-behenyl polyoxyethylene methacrylate), and Sipomer SEM-25 from Rhodia (ω-tristyrylphenyl polyoxyethylene methacrylate),
α-β ethylenically unsaturated monomers which are precursors of hydrophilic units or blocks, such as vinyl acetate, which, once polymerized, can be hydrolyzed in order to give rise to vinyl alcohol units or polyvinyl alcohol blocks,
α-β ethylenically unsaturated monomers of the ureido type, and in particular 2-imidazolidinone-ethyl methacrylamide (Sipomer WAM II from Rhodia).

By way of examples of zwitterionic monomers, mention may be made of:
sulfobetaine monomers, such as sulfopropyldimethylammonium ethyl methacrylate (SPE from Raschig), sulfopropyl dimethylammonium propylmethacrylamide (SPP from Raschig), and sulfopropyl-2-vinylpyridinium (SPV from Raschig),
phosphobetaine monomers, such as phosphatoethyltrimethylammonium ethyl methacrylate.

By way of examples of crosslinking monomers, mention may be made of:
divinylbenzene
ethylene glycol dimethacrylate
allyl methacrylate
methylenebis(acrylamide)
glyoxalbis(acrylamide)
butadiene
triallyl isocyanurate.

The mean molar mass by weight of said polymer (measured by THF gel permeation chromatography (GPC) and expressed in polystyrene equivalents) can be at least 20 000 g/mol, preferably between 50 000 and 1 000 000 g/mol, more preferably of the order of 100 000 to 1 000 000 g/mol.

Said polymers constituting the active material can be obtained in a known manner, preferably by free-radical polymerization, in aqueous medium, of the ethylenically unsaturated monomers. Dispersions of particles of polymers or polymeric dispersions can in particular be obtained by free-radical polymerization in emulsion in water. The polymer solids content can be of the order of 5 to 60% by weight.

Methods for obtaining small-diameter nanoparticulate dispersions are described in Colloid Polym. Sci. 266:462-469 (1988) and in Journal of Colloid and Interface Science, Vol. 89, No. 1, September 1982 pages 185 et seq.

A method of preparation of dispersions of particles having a mean size of less than 100 nm, in particular having a mean size ranging from 1 to 60 nm, most particularly from 5 to 40 nm, is described in EP-A-644 205.

Preferably, the active material is a polymer that derives from monomers, the choice and the relative amounts of which are such that said polymer has a glass transition temperature Tg of the order of −80° C. to +150° C., most particularly of the order of −80° C. to +40° C.

A first embodiment of the invention consists of the use, as an active material, of an organic polymer that is insoluble in the medium of the formulation and in the rinsing medium.

According to the invention, said polymer constituting the active material is considered to be insoluble when less than 15%, preferably less than 10%, of its weight is soluble in the medium of the formulation in the rinsing medium. The content of units derived from potentially anionic monomer that may optionally be present depends on the nature of the other monomers used to prepare the active material; this content is generally less than 10% of the total mass of monomers.

Preferably, said polymer constituting the insoluble active material is a polymer derived from at least one nonionic hydrophobic monomer, or a polymer derived from at least one nonionic hydrophobic monomer and from 0.1 to 20% of its weight of at least one monomer that is potentially cationic in the medium of the formulation.

A second embodiment of the invention consists in using, as an active material, an organic polymer that is insoluble in the medium of the formulation, and that is capable of swelling in the rinsing medium.

Preferably, said polymer capable of swelling derives from at least one nonionic hydrophobic monomer and from 10 to 50% of its weight of at least one monomer that is potentially anionic in the rinsing medium.

As examples of polymers constituting the active material, mention may be made of:
poly(butyl acrylate)s having a Tg of the order of −55° C., and a mean particle size of the order of 50 nm,
butyl acrylate/dimethylaminoethyl methacrylate polymers, with a weight ratio of 98/2, having a Tg of the order of −50° C., and a mean particle size of the order of 60 nm,
butyl acrylate/glyceryl monomethacrylate polymers, with a weight ratio of 95/5, having a Tg of the order of −50° C., and a mean particle size of the order of 50 nm,
butyl acrylate/MAPTAC polymers, with a weight ratio of 95/5, having a Tg of the order of −40° C., and a mean particle size of the order of 50 nm,
butyl acrylate/trimethylammonium methyl methacrylate chloride polymers, with a weight ratio of 95/5, having a Tg of the order of −40° C., and a mean particle size of the order of 50 nm,
   obtained by emulsion polymerization in the presence of 10% by weight of a cationic surfactant such as Dehyquart® ACA from Cognis.

According to one variant, the solid active material in the form of particles contains, encapsulated in its particles, at least one liquid or solid hydrophobic organic active compound that is different from the abovementioned active material.

The hydrophobic active compound is not miscible or is very poorly miscible with water or with a water/alcohol mixture; this means more especially that its water-solubility at pH 7 is less than 20% by weight, preferably less than 10% by weight.

The term “active compound” signifies both an active compound alone or solubilized in a solvent that is not or is poorly miscible in water or a water/alcohol mixture, and a mixture of such compounds, which may or may not be solubilized in a solvent.

As examples of active compounds, mention may in particular be made of mineral or organic oils, fats or waxes of animal or plant origin, and also their derivatives; silicone oils, resins or gums; aromas; essential oils; fragrances; antimicrobial agents; liposoluble vitamins and their derivatives; phospholipids; bactericides; UV-absorbing agents and antidandruff agents; alone or as mixtures. For further details regarding such compounds, reference may be made to the description which will be given below.

Said active compound can be introduced into the particles of the active material in a known manner.

It can be introduced, inter alia, during the actual synthesis of the particles of active material, in particular in the solubilized state in at least one of the monomers from which the active material derives.

If the active compound is liquid and sufficiently “swelling” for the active material, the active compound can also be introduced directly into the preparation of active material obtained by emulsion polymerization; if necessary, a polymer or copolymer swelling “transfer” solvent can be used. As examples of transfer solvents, mention may be made of esters, ketones, alcohols, aliphatic, cycloaliphatic or aromatic hydrocarbons, that are optionally chlorinated, and dialkyl ethers. The transfer solvent can then be eliminated by evaporation.

The amount of active compound present in the particles of active material, if it is present, is usually between 20 and 70 parts, preferably between 40 and 60 parts by weight per 100 parts by weight of active material.

In accordance with a second variant of the invention, the active material is chosen from mineral or organic oils, fats or waxes of animal or plant origin, and their derivatives; silicone oils, resins or gums; aromas; essential oils; fragrances; antimicrobial agents; liposoluble vitamins and their derivatives; phospholipids; bactericides; UV-absorbing agents and antidandruff agents; alone or as mixtures.

As regards the mineral oils, fats and waxes, mention may be made, for example, of petroleum fractions, naphthenic oils, paraffin oils (liquid petroleum jelly), paraffin waxes or microcrystalline waxes. Even though this constitutes a misuse of language, the following are considered to be in this category: compounds of mineral fossil origin, such as, for example, montan, ozokerite, Utah wax; compounds of synthetic origin such as polyethylene, sterone, carbowax, polypropylene or naphthalene.

As organic oils, fats or waxes of animal origin, the following may be suitable: sperm oil, whale oil, seal oil, sardine oil, herring oil, shark oil, cod liver oil; pig fat or sheep fat (tallows); beeswax, lanolin.

By way of examples of organic oils, fats or waxes of plant origin, mention may be made, inter alia, of rapeseed oil, sunflower oil, peanut oil, olive oil, walnut oil, corn oil, soybean oil, linseed oil, hemp oil, grape seed oil, coconut oil, palm oil, cotton seed oil, babassu oil, jojoba oil, sesame oil, castor oil, cacao butter, karite butter, carnauba wax and candelilla wax.

As derivatives, the products derived from the alcoholysis of the abovementioned oils, fats and waxes are first of all denoted.

Among the derivatives, mention may also be made of saturated or unsaturated fatty acids, saturated or unsaturated fatty alcohols, fatty acid esters, or mixtures thereof.

Advantageously, said acids comprise 8 to 40 carbon atoms, more particularly 10 to 40 carbon atoms, preferably 18 to 40 carbon atoms, and can comprise one or more conjugated or unconjugated ethylenic unsaturations and, optionally, one or more hydroxyl groups. As regards the alcohols, they can comprise one or more hydroxyl groups.

As examples of saturated fatty acids, mention may be made of palmitic acid, isostearic acid, stearic acid and behenic acid.

As examples of unsaturated fatty acids, mention may be made of myristoleic acid, palmitoleic acid, oleic acid, erucic acid, linoleic acid, linolenic acid, arachidonic acid and ricinoleic acid, and mixtures thereof.

As regards the alcohols, they comprise more particularly 4 to 40 carbon atoms, preferably 10 to 40 carbon atoms, optionally one or more conjugated or unconjugated ethylenic unsaturations and, optionally, several hydroxyl groups. Polymers comprising several hydroxyl groups may similarly be suitable, such as, for example, polypropylene glycols.

As examples of alcohols, mention may be made, for example, of those corresponding to the abovementioned acids.

As regards the fatty acid esters, they can advantageously be obtained from fatty acids chosen from the abovementioned compounds. The alcohols from which these esters are prepared comprise more particularly 1 to 6 carbon atoms. Preferably, they are methyl, ethyl, propyl or isopropyl esters.

The active material can also be chosen from silicone oils and/or resins and/or gums.

More particularly, the silicone is chosen from polyorganosiloxane oils and/or resins consisting entirely or partly of units having the formulae:
R′₃₋ₐRₐSiO_{1/2} (unit M) and R₂SiO (unit D)

in which formulae:
a is an integer from 0 to 3;
the radicals R, which may be identical or different, represent:
   a saturated or unsaturated, aliphatic hydrocarbon-based group containing from 1 to 15 carbon atoms, preferably from 1 to 10 carbon atoms;
   an aromatic hydrocarbon-based group containing from 6 to 13 carbon atoms;
   a hydrophilic organic group attached to the silicon via an Si—C or Si—O—C bond;
   a hydrogen atom;
the radicals R′, which may be identical or different, represent:
   an OH group;
   an alkoxy or alkenyloxy group containing from 1 to 10 carbon atoms;
   an aryloxy group containing from 6 to 13 carbon atoms;
   an aminofunctional or amidofunctional group containing from 1 to 6 carbon atoms, attached to the silicon via an Si—N bond.

Preferably, the radicals R are chosen from optionally halogenated C₁-C₁₀ alkyl radicals, such as methyl, ethyl, octyl or trifluoropropyl; alkenyl radicals, preferably C₂-C₁₀ alkenyl, such as vinyl allyl, hexenyl, decenyl or decadienyl; aryl radicals, preferably C₆-C₁₃ aryl radicals, such as phenyl.

In relation more especially to the radicals R corresponding to a hydrophilic organic group attached to the silicon via an Si—C or Si—O—C bond, mention may be made of:

hydroxy functional groups, such as alkyl groups substituted with one or more hydroxyl or di(hydroxyalkyl) amino groups and optionally interrupted with one or more divalent hydroxyalkylamino groups. The term “alkyl” is intended to mean a preferably C₁-C₁₀ hydrocarbon-based chain.

Examples of these groups are in particular: —(CH₂)₃—OH; —(CH₂)₄N(CH₂CH₂OH)₂; —(CH₂)₃N(CH₂CH₂OH)—CH₂CH₂—; —N(CH₂CH₂OH)₂; —(CH₂)₃—O—CH₂—CH(OH)—CH₂—N(CH₂—CH₂—NH₂); —(CH₂)₃—O—CH₂—CH (—N (—CH₂—CH₂—NH₂))—CH₂ (OH); —CH(CH₃)—CH₂—O—CH₂—CH(OH)—CH₂—N (—CH₂—CH₂—NH₂); —CH(CH₃)—CH₂—O—CH₂—CH (—NCH₂—CH₂—NH₂))—CH₂ (OH);

aminofunctional groups, such as alkyl substituted with one or more amino or aminoalkylamino groups where alkyl is as defined above, the amino group being primary, secondary, tertiary or ammonium (quaternary), and also with 1,1,6,6-tetrasubstituted derivatives of piperidine.

Examples of a aminofunctional groups are —(CH₂)₃—NH₂; —(CH₂)₃NH(CH₂)₂NH₂.

Relative to the tetrasubstituted derivatives of piperidine, the latter are more particularly substituted, at the 1,1,6,6-positions (i.e. the two carbon atoms of the ring that are adjacent to the nitrogen atom), with a C₁-C₈ alkyl group, and the nitrogen atom of the ring is substituted with a hydrogen, an oxygen, a C₁-C₈ alkyl radical, a hydroxyl radical, a hydroxy(C₂-C₄)alkyl radical, an alkylcarbonyl radical, a phenyl radical, a benzyl radical, or a linear or cyclic C₁-C₆ alkoxy radical. In addition, the piperidine group is advantageously attached to a silicon atom via the carbon in the 3-position, to a radical of formula -Z-R—, in which formula Z represents —(CH₂)ₚ—O—, or —(CH₂)ₚ—NR¹— where R¹ is a hydrogen atom or an alkyl radical comprising 1 to 12 carbon atoms; and where R represents —R²—, —R²CO—, —R³—O—R³, —R³COOR³— or —R³OR³—OCOR³—, in which R² represents a linear or branched alkyl radical comprising 2 to 20 carbon atoms, the R³, which may be identical or different, represent a linear or branched alkyl radical comprising 1 to 12 carbon atoms and optionally carrying a hydroxyl group, and p is between 0 and 10, limits inclusive. Such silicones carrying hindered amine functions are well known (HALS) and are in particular described in patents EP 388321 or EP 665258;

amidofunctional groups, such as alkyl substituted with one or more acylamino groups and optionally interrupted with one or more divalent alkyl-CO—N groups, where alkyl is as defined above and acyl represents alkylcarbonyl.

An example is the group —(CH₂)₃—N(COCH₃)—(CH₂)₂NH(COCH₃);

carboxy functional groups, such as carboxyalkyl optionally interrupted with one or more oxygen or sulfur atoms, where alkyl is as defined above.

An example is the group —CH₂—CH₂—S—CH₂—COOH.

As regards more particularly the hydroxyl radical R indicated in the formulae of the units M and D, said radicals may be initially present in the molecule or else may be the result of the hydrolysis of acyloxy groups containing from 2 to 13 carbon atoms or else ketiminoxy groups containing from 3 to 8 carbon atoms.

More particularly, the acyloxy group is preferably a (C₁-C₁₂) alkylcarbonyl group such as acetoxy; the ketiminoxy group corresponds in particular to ON═C(CH₃)C₂H₅.

Advantageously, at least 80% of the radicals R of said silicones represent a methyl group.

Preferably, the radicals R′ can be chosen from C₁-C₁₀, preferably C₁-C₈, alkoxy radicals, such as methoxy, ethoxy, propoxy, butoxy or octyloxy; C₂-C₁₀, preferably C₂-C₆, alkenyloxy radicals; and C₆-C₁₃ aryloxy radicals, such as phenyloxy.

By way of concrete examples of “units D”, mention may be made of: (CH₃)₂SiO; CH₃(CH═CH₂)SiO; CH₃ (C₆H₅)SiO; (C₆H₅)₂SiO; CH₃HSiO; CH₃ (CH₂—CH₂—CH₂OH)SiO.

By way of concrete examples of “units M”, mention may be made of: (CH₃)₃SiO_{1/2}; (CH₃)_{1/2} (CH═CH₂) SiO_{1/2}; (CH₃)₂HSiO_{1/2}; (OCH₃)₃SiO_{1/2}. [O—C(CH₃)═CH₂] ₃SiO_{1/2}; [ON═C(CH₃)]₃SiO_{1/2}.

Moreover, the silicone used may optionally comprise preferably less than 5% of the units of formula T or Q below:
RSiO_{3/2} (unit T) and/or SiO_{4/2} (unit Q)
in which formula R has the definition given previously.

By way of concrete examples of “units T”, mention may be made of: CH₃SiO_{3/2}; (CH═CH₂)SiO_{3/2}; HSiO_{3/2}.

It should nevertheless be noted that, when the silicones contain reactive radicals R (such as H, vinyl, allyl, hexenyl, etc.), the latter generally represent not more than 5% of the weight, and preferably not more than 1% of the weight, of the silicone.

Among the preferred silicones, mention may be made of silicones of the polydimethylsiloxane (dimethicone), diphenyl dimethicone, phenyl trimethicone, dimethiconol or amodimethicone type, alone or combined.

The active material can be chosen from aromas; essential oils; fragrances, such as, for example, oils and/or essences of mint, of green mint, of peppermint, of menthol, of vanilla, of cinnamon, of laurol, of aniseed, of eucalyptus, of thyme, of sage, of cedar leaf, of nutmeg, of citrus (lemon, lime, grapefruit or orange) or of fruit (apple, pear, peach, cherry, plum, strawberry, raspberry, apricot, pineapple, grape, etc.), alone or as mixtures. Use may also be made of compounds such as benzaldehyde, isoamyl acetate, ethyl butyrate, etc.

The antimicrobial agents can be chosen from thymol, menthol, triclosan, 4-hexylresorcinol, phenol, eucalyptol, benzoic acid, benzoic peroxide, butyl paraben, and mixtures thereof.

As regards the liposoluble vitamins and their derivatives, those that are suitable are in particular vitamin A and its derivatives, for example its esters such as acetate, palmitate or propionate, vitamin B2, pantothenic acid, vitamin D and vitamin E, vitamin C derivatives, such as the esters, in particular the acetate, the propionate or the palmitate, and phospholipids.

Bactericides, for instance triclosan, and antidandruff agents, for instance zinc pyrithione or octopyrox, can also be used as active material.

The active material can also be chosen from UV-absorbing agents, such as aminobenzoate derivatives of the PABA and PARA type, salicylates, cinnamates, anthranilates, dibenzoylmethanes, camphor derivatives, and mixtures thereof.

According to this embodiment, the active material is in the form of liquid or solid particles dispersed in the medium of the formulation. More particularly, the mean diameter of the particles is between 10 nm and 10 μm, preferably between 10 nm and 1 μm.

In accordance with a variant, the active material is combined with at least one liquid or solid hydrophobic organic active compound that is different from the abovementioned active material.

Said hydrophobic organic active compound is advantageously chosen from mineral or organic oils, fats or waxes of animal or plant origin, and their derivatives; silicone oils, resins or gums; aromas; essential oils; fragrances; antimicrobial agents; liposoluble vitamins and their derivatives; phospholipids; bactericides; UV-absorbing agents and antidandruff agents; alone or as mixtures.

The information indicated above with respect to these compounds remains valid and will not be repeated here.

Very advantageously, the liquid or solid hydrophobic organic active compound is a fragrance, a moisturizing or repairing agent, a silicone oil, optionally an amino silicone oil, or a mineral or plant oil.

The amount of active compound present in the active material, if it is present, is usually between 20 and 70 parts, preferably between 40 and 60 parts by weight per 100 parts by weight of active material.

Finally, it should be noted that the use of a formulation comprising several active materials, corresponding to one and/or other of the variants, is not excluded.

Whatever the variant selected, the active material is made into a stable dispersion in the medium of the formulation by means of a cationic and/or nonionic surfactant (stabilizing surfactant).

More particularly, if the active material possesses cationic charges in the medium of the formulation, then the stabilizing surfactant may be either one or more nonionic surfactants, or one or more cationic surfactants, or a mixture of these two types of surfactants.

Moreover, when the material is in a nonionic form in the medium of the formulation, then the stabilizing surfactant advantageously comprises one or more cationic surfactants, optionally combined with one or more nonionic surfactants.

It is specified, most particularly according to this second possibility, that, when the stabilizing surfactant comprises one or more nonionic surfactants, their content advantageously represents less than 70% of the weight of all the surfactants, preferably less than 50% of the weight of all the surfactants.

Preferably, the stabilizing surfactant comprises one or more cationic surfactants and does not contain any nonionic surfactant.

When the active material is at least chosen from polymers, the cationic surfactant present in the formulation can be used at least in part during the synthesis of the polymer constituting the active material.

For the good implementation of the invention, the weight amount of cationic and/or nonionic surfactant in the formulation is more particularly less than or equal to 25% by weight of the formula, preferably less than or equal to 5%.

When the active material consists at least of a polymer, the cationic charges, brought about by the possible cationic or potentially cationic units of the active material and possibly by the cationic surfactant(s), at the surface of the active material dispersed in the medium of the formulation, are such that the zeta potential of the active material dispersed in the medium of the formulation (MAV) is from 0 to +50 mV, preferably from +10 to +40 mV.

Among the cationic surfactants, mention may in particular by made of the quaternary ammonium salts of formula
R¹R²R³R⁴N+X⁻
where:
R¹, R² and R³, which may be similar or different, represent H or an alkyl group containing fewer than 4 carbon atoms, preferably 1 or 2 carbon atom(s), optionally substituted with one or more hydroxyl function(s), or can form, together with the nitrogen atom N⁺, at least one aromatic or heterocyclic ring,
R⁴ represents a C₈-C₂₂, preferably C₁₂-C₂₂ alkyl or alkenyl group, or an aryl or benzyl group, and
X⁻ is a solubilizing anion, such as halides (for example, chloride, bromide or iodide), sulfates or alkyl sulfates (methyl sulfate), carboxylates (acetate, propionate, benzoate), alkylsulfonates or arylsulfonates.

Mention may in particular be made of dodecyltrimethylammonium bromide, tetradecyltrimethylammonium bromide, cetyltrimethylammonium bromide, stearylpyridinium chloride, Rhodaquat® TFR and Rhodamine® C15 sold by Rhodia, cetyltrimethylammonium chloride (Dehyquart ACA and/or AOR from Cognis), or cocobis(2-hydroxyethyl)ethylammonium chloride (Ethoquad C12 from Akso Nobel).

Mention may also be made of other cationic surfactants having softening properties, such as:
quaternary ammonium salts of formula
   R^{1′}R^{2′}R^{3′}R^{4′}N⁺X⁻
   where:
R^{1′} and R^{2′} which may be similar or different, represent H or an alkyl group containing fewer than 4 carbon atoms, preferably 1 or 2 carbon atom(s), optionally substituted with one or more hydroxyl function(s), or can form, together with the nitrogen atom N⁺, a heterocyclic ring,
R^{3′} and R^{4′} represent a C₈-C₂₂, preferably C₁₀-C₂₂ alkyl or alkenyl group, or a aryl or benzyl group, and
X⁻ is an anion such as halides (for example, chloride, bromide or iodide), sulfates or alkyl sulfates (methyl sulfate), carboxylates (acetate, propionate, benzoate), alkylsulfonates or arylsulfonate.

Mention may in particular be made of the dialkyldimethylammonium chlorides, such as ditallow dimethyl ammonium chloride, or methyl sulfates, etc., alkylbenzyldimethylammonium chlorides;
C₁₀-C₂₅ alkylimidazolium salts, such as C₁₀-C₂₅ akylimidazolinium methyl sulfates;
substituted polyamine salts, such as N-tallow-N,N′,N′-triethanol-1,3-propylenediamine dichloride or dimethyl sulfate, N-tallow-N,N,N′,N′,N′-pentamethyl-1,3-propylenediamine dichloride.

Among the nonionic surfactants that may be present, mention may be made of polyoxyalkylenated derivatives such as:
ethoxylated or ethoxy-propoxylated fatty alcohols
ethoxylated or ethoxy-propoxylated triglycerides
ethoxylated or ethoxy-propoxylated fatty acids
ethoxylated or ethoxy-propoxylated sorbitan esters
ethoxylated or ethoxy-propoxylated fatty amines
ethoxylated or ethoxy-propoxylated di(phenyl-1-ethyl)phenols
ethoxylated or ethoxy-propoxylated tri(phenyl-1-ethyl)phenols
ethoxylated or ethoxy-propoxylated alkyl phenols.

Among the elements constituting the formulation, there is at least one carrier agent capable of bringing the active material to the surface of the keratinous fibers during the rinsing process.

According to the invention, said carrier agent:
is an organic polymer that is soluble or dispersible in the medium of the formulation and in the rinsing medium,
has, in the medium of the formulation, an overall ionic charge that is zero or cationic,
is capable of developing, at the pH of the rinsing process in the rinsing medium, a sufficient number of anionic charges to destabilize the active material in the rinsing medium.

Said organic polymer constituting the carrier agent may be any polymer that is soluble or dispersible in an aqueous or aqueous-alcoholic medium having a pH of between 3 and 8, preferably between 4.5 and 8, comprising at least one unit that is neutral in the medium of the formulation and potentially anionic in the rinsing medium.

They may also comprise at least one unit that is cationic or potentially cationic in the medium of the formulation and/or at least one hydrophilic or hydrophobic, nonionic unit.

The term “dispersible” means that the carrier agent does not form any macroscopic precipitate in an aqueous or aqueous-alcoholic medium.

Preferably, the polymer constituting the carrier agent is a polymer comprising:
at least one hydrophilic unit that is neutral in the medium of the formulation and potentially anionic in the rinsing medium, and
at least one hydrophilic unit that is cationic or potentially cationic in the medium of the formulation,
and, optionally, at least one hydrophilic or hydrophobic, nonionic unit.

The polymer constituting the carrier agent can optionally contain anionic units (the first pKa of which is less than 3), but this is in a very small amount, for example in an amount much less than 5% by weight relative to all the units.

The relative amounts of the various units of the polymer constituting the carrier agent are such that, in the medium of the formulation, the overall charge of the carrier agent is zero or cationic.

The relative amounts of carrier agent polymer, of cationic and/or nonionic surfactant and of active material are such that, during the rinsing process, the number of anionic charges developed in the rinsing medium by the carrier agent is sufficient to destabilize the active material in the rinsing medium. This destabilization may be due, for example, to the electrostatic attraction with the surface charges of the active material in the medium.

According to the invention, the active material is considered to be destabilized in the rinsing medium comprising the carrier agent, when the turbidity of said medium reaches, in less than 5 minutes, a value at least 5 times greater than the turbidity that the same medium would have in the absence of carrier agent.

The number of anionic charges developed in the rinsing medium by the carrier agent so as to destabilize the active material is preferably at least 1% relative to the number of surface cationic charges of the active material in the medium.

Moreover, this number of anionic charges preferably remains at most 200% relative to the number of surface cationic charges of the active material in the medium.

As examples of polymers that may constitute the carrier agent, mention may in particular be made of polymers derived from ethylenically unsaturated monomers, and also natural polysaccharides and substituted or modified polysaccharides, taken alone or as a mixture with one another.

A first example of polymers that may constitute the carrier agent are polymers derived:
from at least one α-β monoethylenically unsaturated monomer that is neutral in the medium of the formulation and potentially anionic in the rinsing medium, and
optionally from at least one α-β monoethylenically unsaturated monomer that is cationic or potentially cationic in the medium of the formulation, and
optionally from at least one hydrophilic or hydrophobic, preferably hydrophilic, nonionic α-β monoethylenically unsaturated monomer.

Preferably, the carrier agent is a random, block or grafted polymer derived:
from at least one α-β monoethylenically unsaturated hydrophilic monomer that is neutral in the medium of the formulation and potentially anionic in the rinsing medium, and
from at least one α-β monoethylenically unsaturated hydrophilic monomer that is cationic or potentially cationic in the medium of the formulation,
and, optionally, from at least one hydrophilic or hydrophobic, preferably hydrophilic nonionic α-β monoethylenically unsaturated monomer.

The relative amounts of monomers from which the carrier agent derives are such that, in the medium of the formulation, the overall charge of the copolymer constituting the carrier agent is zero or cationic.

The mean molar mass by weight of the carrier agent derived from one or more α-β monoethylenically unsaturated monomers (measured by aqueous gel permeation chromatography (GPC) and expressed in polyoxyethylene equivalents) is greater than 5000 g/mol, generally of the order of 20 000 to 500 000 g/mol.

As examples of hydrophilic α-β monoethylenically unsaturated monomers that are neutral in the medium of the formulation and potentially anionic in the rinsing medium, mention may be made of:
monomers having at least one carboxylic function, for instance α-β ethylenically unsaturated carboxylic acids or the corresponding anhydrides, such as acrylic, methacrylic or maleic acids or anhydrides, fumaric acid, itaconic acid, N-methacroylalanine, N-acryloylglycine, and their water-soluble salts,
monomers which are precursors of carboxylate functions such as tert-butyl acrylate, which, after polymerization, give rise to carboxylic functions by hydrolysis.

As examples of hydrophilic α-β monoethylenically unsaturated monomers that are cationic or potentially cationic in the medium of the formulation, mention may be made of:
acryloyl- or acryloyloxyammonium monomers such as trimethylammonium propyl methacrylate chloride, trimethylammonium ethylacrylamide or -methacrylamide chloride or bromide, trimethylammoniumbutylacrylamide or -methacrylamide methyl sulfate, trimethylammonium propylmethacrylamide methyl sulfate (MES), (3-methacrylamidoproyl)trimethylammonium chloride (MAPTAC), (3-acrylamidopropyl)trimethylammonium chloride (APTAC), methacryloyloxyethyltrimethylammonium chloride or methyl sulfate, and acryloyloxyethyltrimethylammonium chloride;
1-ethyl-2-vinylpyridinium or 1-ethyl-4-vinylpyridinium bromide, chloride or methyl sulfate;
N,N-dialkyldiallylamine monomers such as N,N-dimethyldiallylammonium chloride (DADMAC);
polyquaternary monomers such as dimethylaminopropylmethacrylamide chloride and N-(3-chloro-2-hydroxypropyl)trimethylammonium (DIQUAT), etc.,
carboxybetaine monomers,
N,N-(dialkylamino-ω-alkyl)amides of α-β monoethylenically unsaturated carboxylic acids such as N,N-dimethylaminoethylacrylamide or -methacrylamide, 2-(N,N-dimethylamino)ethylacrylamide or -methacrylamide, 3-(N,N-dimethylamino) propylacrylamide or -methacrylamide, and 4-(N,N-dimethylamino)butylacrylamide or -methacrylamide,
α-β monoethylenically unsaturated amino esters such as 2-(dimethylamino)ethyl methacrylate (DMAM), 3-(dimethylamino)propyl methacrylate, 2-(tert-butyl-amino)ethyl methacrylate, 2-(dipentylamino)ethyl methacrylate, and 2-(diethylamino)ethyl methacrylate,
monomers which are precursors of amine functions, such as N-vinylformamide, N-vinylacetamide, etc., which give rise to primary amine functions by simple acid or base hydrolysis.

As examples of hydrophilic α-β monoethylenically unsaturated monomers that are uncharged or non-ionizable, mention may be made of:
hydroxyalkyl esters of α-β ethylenically unsaturated acids, such as hydroxyethyl and hydroxypropyl acrylate and methacrylate, glyceryl monomethacrylate, etc.,
α-β ethylenically unsaturated amides such as acrylamide, N,N-dimethylmethacrylamide, N-methylolacrylamide, etc.,
α-β ethylenically unsaturated monomers bearing a water-soluble polyoxyalkylene segment of the polyethylene oxide type, such as polyethylene oxide α-methacrylates (Bisomer S20W, S10W, etc., from Laporte) or α,ω-dimethacrylates, Sipomer BEM from Rhodia (ω-behenyl polyoxyethylene methacrylate), and Sipomer SEM-25 from Rhodia (ω-tristyrylphenyl polyoxyethylene methacrylate),
α-β ethylenically unsaturated monomers which are precursors of hydrophilic units or blocks, such as vinyl acetate, which, once polymerized, can be hydrolyzed in order to give rise to vinyl alcohol units or polyvinyl alcohol blocks,
α-β ethylenically unsaturated monomers of the ureido type, and in particular 2-imidazolidinone-ethyl methacrylamide (Sipomer WAM II from Rhodia).

By way of examples of hydrophobic nonionic α-β monoethylenically unsaturated monomers, mention may be made of:
vinylaromatic monomers such as styrene, vinyltoluene, etc.,
alkyl esters of α-β monoethylenically unsaturated acids, such as methyl, ethyl, etc. acrylates and methacrylates,
vinyl esters or allyl esters of saturated carboxylic acids, such as vinyl or allyl acetates, propionates or versatates,
α-β monoethylenically unsaturated nitriles such as acrylonitrile, etc.

As examples of anionic hydrophilic α-β monoethylenically unsaturated monomers (the first pKa of which is less than 3), mention may be made of:
monomers having at least one sulfate or sulfonate function, such as 2-sulfoxyethyl methacrylate, vinylbenzene sulfonic -acid, allyl sulfonic acid, 2-acrylamido-2-methylpropane sulfonic acid, sulfoethyl acrylate or methacrylate, sulfopropyl acrylate or methacrylate, and their water-soluble salts,
monomers having at least one phosphonate or phosphate function, such as vinylphosphonic acid, etc., the esters of ethylenically unsaturated phosphates, such as the phosphates derived from hydroxyethyl methacrylate (Empicryl 6835 from Rhodia) and those derived from polyoxyalkylene methacrylates, and their water-soluble salts.

By way of examples of polymers derived from ethylenically unsaturated monomers constituting the carrier agent, mention may be made of:
polyacrylic or polymethacrylic acids, alkali metal polyacrylates or polymethacrylates, preferably with a mean molar mass by weight of 100 000 to 1 000 000 g/mol,
acrylic acid/DADMAC polymers, having a molar ratio of 50/50 to 30/70, preferably with a mean molar mass by weight of 70 000 to 350 000 g/mol,
acrylic acid/MAPTAC polymers, having a molar ratio of 60/40 to 30/70, preferably with a mean molar mass by weight of 90 000 to 300 000 g/mol,
acrylic acid/MAPTAC/linear C₄-C₁₈ alkyl methacrylate polymers comprising from 0.005 to 10% by mass of alkyl methacrylate, with an acrylic acid/MAPTAC molar ratio ranging from 60/40 to 30/70, and preferably with a mean molar mass by weight of 50 000 to 250 000 g/mol,
acrylic acid/dimethylaminoethyl methacrylate (DMAEMA) polymers, with a molar ratio of 60/40 to 30/70, preferably with a mean molar mass by weight of 50 000 to 300 000 g/mol.

A second example of polymers that can constitute the carrier agent are potentially anionic natural polysaccharides and potentially anionic or amphoteric, substituted or modified polysaccharides.

The potentially anionic natural polysaccharides are formed of nonionic monosaccharide units and of monosaccharide units that are neutral in the medium of the formulation and potentially anionic in the rinsing medium, these units being similar or different. They may be linear or branched.

More particularly, said potentially anionic natural polysaccharides are branched polysaccharides formed:
of a main chain comprising anhydrohexose units that may be similar or different,
and of branches comprising at least one anhydropentose and/or anhydrohexose unit that is neutral in the medium of the formulation and optionally potentially anionic in the rinsing medium.

The (similar or different) hexose units of the main chain may be D-glucose, D- or L-galactose, D-mannose, D- or L-fucose or L-rhamnose units.

The (similar or different) pentose and/or hexose units that are nonionic or neutral in the medium of the formulation and potentially anionic in the rinsing medium, of the branches, may be units of D-xylose, etc., L- or D-arabinose, D-glucose, D- or L-galactose, D-mannose, D- or L-fucose, L-rhamnose, D-glucuronic acid, D-glacturonic acid, D-mannuronic acid, D-mannose substituted with a pyruvic group, etc.

By way of examples of natural polysaccharides that are neutral in the medium of the formulation and potentially anionic in the rinsing medium, mention may be made of xanthan gums (such as Rhodopol® from Rhodia), succinoglycans, rhamsans, gellan gums, welan gums, etc.

Their mean molar mass by weight is more particularly between 2000 and 5 000 000 g/mol, preferably from 10 000 to 5 000 000 g/mol, most particularly from 10 000 to 4 000 060 g/mol. The mean molar mass by weight Mw of said polysaccharides may be measured by size exclusion chromatography.

When the polysaccharides in question are substituted or modified, their natural backbone is formed of nonionic monosaccharide units and/or of monosaccharide units that are neutral in the medium of the formulation and potentially anionic in the rinsing medium, said monosaccharide units being similar or different, and being substituted or modified:
with one or more group(s) which carries or carry at least one charge that is neutral in the medium of the formulation and potentially anionic in the medium,
and optionally with one or more group(s) which carry or carries at least one charge that is cationic or potentially cationic in the medium of the formulation,
   the degree of substitution or of modification of the monosaccharide units with the entirety of the groups which carry charges that are potentially anionic and of optional groups which carry cationic charges being such that said substituted or modified polysaccharide is soluble or dispersible in an aqueous or aqueous-alcoholic medium and has an overall zero or cationic charge in the medium of the formulation.

Said substituted or modified polysaccharides may also contain at least one nonionic modifying or substituent group.

Among the natural backbones that can be used, mention may be made of linear or branched polysaccharides.

More particularly, said polysaccharide is a substituted or modified branched polysaccharide, the natural backbone of which is formed:
from a main chain comprising similar or different anhydrohexose units,
and from branches comprising at least one anhydropentose and/or anhydrohexose unit that is neutral in the medium of the formulation and optionally potentially anionic in the rinsing medium,
   the anhydrohexose and/or anhydropentose units of said polysaccharide being substituted or modified with one or more groups which carry at least one charge that is neutral in the medium of the formulation and potentially anionic in the rinsing medium, and optionally at least one charge that is cationic or potentially cationic in the rinsing medium,
   the degree of substitution or of modification DSi of the anhydrohexose and/or anhydropentose units with the entirety of said groups which carry charges that are ionic or potentially ionic ranging from 0.01 to less than 3, preferably from 0.01 to 2.5,
   with a ratio of the number of charges that are potentially anionic in the rinsing medium to the number of charges that are cationic or potentially cationic in the medium of the formulation ranging from 100/0 to 30/70, preferably from 100/0 to 50/50.

When the polysaccharide in question is an amphoteric polysaccharide, the ratio of the number of charges that are potentially anionic in the rinsing medium to the number of charges that are cationic or potentially cationic in the medium of the formulation ranges from 99.5/0.5 to 30/70, preferably from 99.5/0.5 to 50/50.

Said substituted or modified branched polysaccharide may also contain at least one nonionic modifying or substituting group.

The mean molar mass by weight of said substituted or modified polysaccharides can range from 2000 to 5 000 000 g/mol, preferably from 10 000 to 5 000 000 g/mol.

The mean molar mass by weight Mw of said polysaccharides can be measured by size exclusion chromatography.

When the polysaccharide in question is a polysaccharide carrying substituent groups that are potentially anionic in the rinsing medium, the measurement is made in water, at pH 9-10, containing 0.1 M of LiCl and 2/10 000 of sodium nitrate.

When the polysaccharide in question is an amphoteric polysaccharide, i.e. carries substituent groups that are potentially anionic in the rinsing medium and groups that are cationic or potentially cationic in the medium of the formulation, the measurement is made in a 0.1 M aqueous solution of formic acid containing 0.05 M of sodium nitrate and 10 ppm of high molar mass polyallyldimethylamine chloride (PDADMA) in the case of polysaccharides for which the DSi in terms of ionic or potentially ionic function is less than 0.5. For those for which the DSi is greater than 0.5, a 0.025 M aqueous hydrochloric acid solution is used.

The mean molar mass by weight Mw is established directly in the known manner by means of the light scattering values.

The degree of substitution or of modification, DSi, corresponds to the average number of hydroxyl functions of the anhydrohexose and/or anhydropentose units that are substituted or modified with said ionic or potentially ionic group(s), per anhydrohexose and/or anhydropentose unit.

Said ionic or potentially ionic groups are attached to the carbon atoms of the sugar backbone either directly or by means of —O— bonds.

In the case of amphoteric polysaccharides, the potentially anionic charges may be provided by substituent groups or modifying groups that are different from those carrying cationic or potentially cationic charges; said polymer is then an ampholyte polysaccharide.

When the same substituent group or modifying group carries both a potentially anionic charge and a cationic or potentially cationic charge, said polysaccharide is then of betaine type.

Said substituted or modified polysaccharide may also exhibit at least one nonionic modifying group or substituent group. Said nonionic groups are attached to the carbon atoms of the sugar backbone either directly or by means of —O— bonds. The presence of such groups is expressed in number of moles of substitution MS, i.e. in average number of moles of precursor of said nonionic substituent which have reacted per anhydrohexose and/or anhydropentose unit.

If said precursor is not able to form new reactive hydroxyl groups (alkylation precursor, for example), the degree of substitution or of modification with the entirety of the ionic or ionizable groups and nonionic groups is less than 3 by definition.

If said precursor is capable of forming new reactive hydroxyl groups (hydroxyl alkylation precursor, for example), there is theoretically no limit on the number of moles of substitution MS; it may, for example, be up to 6, preferably up to 2.

Among the groups that are potentially anionic in the rinsing medium, mention may be made of those containing one or more carboxylate (carboxylic) functions.

Mention may in particular be made of those of formula:
—[—CH₂—CH(R)—O]—(CH₂)_{y}—COOH or
—[—CH₂—CH(R)—O]ₓ— (CH₂)_{y}—COOM
where
R is a hydrogen or an alkyl radical containing from 1 to 4 carbon atoms
x is an integer ranging from 0 to 5
y is an integer ranging from 0 to 5
M represents an alkali metal.

Mention may most particularly be made of the carboxy groups —COO⁻Na⁺ attached directly to a carbon atom of the sugar backbone, and of carboxymethyl (sodium salt) groups —CH₂—COO⁻Na⁺ attached to a carbon atom of the sugar backbone by means of an —O— bond.

Among the cationic or potentially cationic groups, mention may be made of those containing one or more amino, ammonium, phosphonium or pyridinium functions.

Mention may in particular be made of the cationic or potentially cationic groups of formula:
—NH₂
—[—CH₂—CH(R)—O]ₓ—(CH₂)_{y}—COA-R′—N(R″)₂
—[—CH₂—CH(R)—O]ₓ—(CH₂)_{y}—COA-R′—N⁺(R′″)₃ X⁻
—[—CH₂—CH(R)—O]ₓ—(CH₂)_{y}—COA-R′—NH—R′″—N(R″)₂
—[—CH₂—CH(R)—O]ₓ—R′—N(R″)₂
—[—CH₂—CH(R)—O]ₓ—R′—N⁺(R′″)₃ X⁻
—[—CH₂—CH(R)—O]ₓ—R′—NH—R″″—N(R″))₂
—[—CH₂—CH(R)—O]ₓ—Y—R″
where
R is a hydrogen atom or an alkyl radical containing from 1 to 4 carbon atoms,
x is an integer ranging from 0 to 5,
y is an integer ranging from 0 to 5,
R′ is an alkylene radical containing from 1 to 12 carbon atoms, optionally carrying one or more substituents OH,
the radicals R″, which may be similar or different, represent a hydrogen atom or an alkyl radical containing from 1 to 18 carbon atoms,
the radicals R′″, which may be similar or different, represent an alkyl radical containing from 1 to 18 carbon atoms,
R″″ is a linear, branched or cyclic alkylene radical containing from 1 to 6 carbon atoms,
A represents O or NH,
Y is a heterocyclic aliphatic group comprising from 5 to 20 carbon atoms and a nitrogen hetero atom,
X⁻ is a counterion, preferably halide (chloride, bromide, iodide, in particular),
   and also N-alkylpyridinium-yl groups in which the alkyl radical contains from 1 to 18 carbon atoms, with a counterion, preferably of halide type (chloride, bromide, iodide, in particular).

Among the cationic or potentially cationic groups, mention may be made most particularly of:

those of formula
—NH₂
—CH₂—CONH—(CH₂)₂—N(CH₃)₂
—CH₂—COO—(CH₂)₂—NH—(CH₂)₂—N(CH₃)₂
—CH₂—CONH—(CH₂)₃—NH—(CH₂)₂—N(CH₃)₂
—CH₂—CONH—(CH₂)₂—NH—(CH₂)₂—N(CH₃)₂
—CH₂—CONH—(CH₂)₂—N⁺(CH₃)₃Cl⁻
—CH₂—CONH—(CH₂)₃—N⁺(CH₃)₃Cl⁻
—(CH₂)₂—N(CH₃)₂
—(CH₂)₂—NH—(CH₂)₂—N(CH₃)₂
—(CH₂)₂—N₊(CH₃)₃ C⁻
2-hydroxypropyltrimethylammonium chloride —CH₂—CH(OH)—CH₂—N⁺(CH₃)₃Cl⁻

pyridinium-yl groups such as N-methylpyridinium-yl, of formula 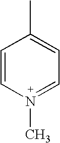
with a chloride counterion

hindered amino groups such as those derived from HALS amines of general formula:

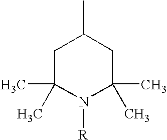

where R represents CH₃ or H.

Among the betaine groups, mention may most particularly be made of the functional formula: —(CH₂)₂—N⁺(CH₃)₂—(CH₂)₂—COO—, an ethyldimethylammonium betaine function.

Among the nonionic groups, mention may be made of those of formula:
—[—CH₂—CH(R)—O]ₓ—R¹ where
R is a hydrogen atom or an alkyl radical containing from 1 to 4 carbon atoms,
x is an integer ranging from 0 to 5,
R¹ represents
   a hydrogen atom,
   an alkyl radical containing from 1 to 22 carbon atoms, optionally interrupted with one or more oxygen and/or nitrogen hetero atoms, or cycloalkyl, aryl or arylalkyl, containing from 6 to 12 carbon atoms,
   a radical —(CH₂)_{y}—COOR
   a radical —(CH₂)_{y}—CN,
   a radical —(CH₂)_{y}—CONHR²,
R² representing an alkyl, aryl or arylalkyl radical containing from 1 to 22 carbon atoms,
and y is an integer ranging from 0 to 5,
   —CO—NH—R¹
R¹ having the definition given above, attached to a carbon atom of the sugar backbone by means of an —O— bond.

Mention may most particularly be made of the following groups:
methyl, ethyl, propyl, isopropyl, butyl, hexyl, octyl, dodecyl, octadecyl, phenyl, benzyl, attached to a carbon atom of the sugar backbone by means of an ether, ester, amide or urethane linkage,
cyanoethyl, hydroxyethyl, hydroxypropyl, hydroxybutyl, attached to a carbon atom of the sugar backbone by means of an —O— bond.

The (similar or different) hexose units of the main chain of the natural backbone may be units of D-glucose, D- or L-galactose, D-mannose, D- or L-fucose, L-rhamnose, etc.

The (similar or different) pentose and/or hexose units that are nonionic or neutral in the medium of the formulation and potentially anionic in the rinsing medium, of the branches of the natural backbone, may be units of D-xylose, L- or D-arabinose, D-glucose, D- or L-galactose, D-mannose, D- or L-fucose, L-rhamnose, D-glucuronic acid, D-galacturonic acid or D-mannuronic acid.

By way of examples of natural backbone, mention may be made of galactomannans, galactoglucomannans, xyloglucans, xanthan gums, scleroglucanns, succinoglycans, rhamsans, welan gums, etc.

Preferably, the natural backbone is a galactomannan.

Galactomannans are macromolecules containing a main chain of D-mannopyranose units linked in the β(1-4) position, which is substituted with D-galactopyranose units in the α(1-6) position. Among these, mention may be made of guar gum, carob gum and tara gum.

Preferably, the natural backbone is a guar gum. Guar gums have a mannose/galactose ratio of 2.

The substituted or modified polysaccharides used according to the invention can be obtained by functionalization of the natural backbone by means of precursors of the ionic or potentially ionic groups and optionally nonionic groups.

These functionalization operations can be carried out in the known manner by oxidation, substitution, condensation and/or addition.

According to a preferred embodiment, the natural backbone of said substituted or modified polysaccharide is a galactomannan.

By way of examples of substituted or modified polysaccharides that can be used according to the invention, mention may be made of:
carboxymethylgalactomannans, in particular carboxymethylguars,
carboxymethylhydroxypropylgalactomannans, in particular carboxymethylhydroxypropylguars,
carboxymethylhydroxypropyltrimethylammonium chloride galactomannans, in particular carboxymethylhydroxypropyltrimethylammonium chloride guars,
carboxymethylhydroxypropyl-hydroxypropyltrimethylammonium chloride galactomannans, in particular carboxymethylhydroxpropyl-hydroxypropyltrimethylammonium chloride guars.

When the carrier agent is a polysaccharide, it is preferable for the dispersion of active material in the medium of the formulation comprising the carrier agent to have a pH of between 3 and 5.5, preferably of between 4.5 and 5.5 when said polysaccharide is a substituted or modified guar.

For effective realization of the invention, the amount of carrier agent present in the formulation according to the invention is more particularly between 0.001 and 50 parts by weight, preferably from 0.01 to 5 parts, most particularly from 0.05 to 2 parts by weight per 100 parts by weight of active material.

As mentioned previously, the formulation according to the invention can optionally comprise at least one soluble salt.

More particularly, the salt is chosen from chlorides, bromides, iodides, nitrates, sulfates and sulfonates of an alkali metal, such as sodium, or of ammonium (more particularly of NR₄⁺ type, the Rs, which may be identical or different, representing a hydrogen atom or a methyl radical), alone or as mixtures.

The content of the salt in the formulation, if it is present, is less than or equal to 2% by weight of the formula.

The formulation according to the invention may be in the form of a stable dispersion (liquid, cream, paste, gel, etc.).

When the active material is chosen from the polymers, the formulation according to the invention can be obtained by carrying out the following steps:
1) preparation of an aqueous dispersion of active material by aqueous emulsion polymerization in the presence of a cationic and/or nonionic surfactant as stabilizing agent; the solids content of the dispersion obtained may be of the order of 5 to 60% by weight;
2) optional dilution with water or a water/alcohol mixture (depending on the desired proportion of active material in the formulation) and, if necessary, adjustment of the pH to a value of between 3 and 5.5, preferably of between 4.5 and 5.5, using an acid (hydrochloric acid, citric acid, phosphoric acid, benzoic acid, etc.);
3) addition of the carrier agent to the dispersion obtained;
4) optionally, addition of a further amount of cationic and/or nonionic surfactant before or after addition of said carrier agent, and
5) if necessary, readjustment of the pH to a value of between 3 and 5.5, preferably of between 4.5 and 5.5, using an acid.

In the polymerization step 1), the surfactant(s) is (are) preferably cationic.

When the active material is chosen from organic or mineral oils, fats and waxes, silicone oils, resins or gums, essential oils, fragrances, etc., the formulation according to the invention can be obtained by carrying out the following steps:
1) preparation of an aqueous or aqueous-alcoholic dispersion of active material in the presence of a cationic and/or nonionic surfactant as stabilizing agent;
2) optional dilution with water or a water/alcohol mixture (depending on the desired proportion of active material in the formulation) and, if necessary, adjustment of the pH to a value of between 3 and 5.5, preferably of between 4.5 and 5.5, using an acid (hydrochloric acid, citric acid, phosphoric acid, benzoic acid, etc.);
3) addition of the carrier agent to the dispersion obtained;
4) optionally, addition of a further amount of the cationic and/or nonionic surfactant before or after addition of said carrier agent, and
5) if necessary, readjustment of the pH to a value of between 3 and 5.5, preferably of between 4.5 and 5.5, using an acid.

For effective realization of the invention, the aqueous or aqueous-alcoholic formulation comprises, per 100 parts of its weight:
from 0.01 to 50, preferably from 0.05 to 30, parts by dry weight of active material,
from 0.01 to 35, preferably from 0.01 to 20, parts by dry weight of cationic surfactant,
from 0.001 to 5, preferably from 0.01 to 1, part by dry weight of carrier agent,
at most 2 parts by weight of soluble salt.

Said dispersion may have a solids content of 0.01 to 90% by weight, preferably of 0.05 to 50% by weight, more particularly of 0.1 to 5% by weight.

The formulation may also comprise other constituents that are usual in formulations intended to be applied to keratinous material, such as the skin and the hair, and intended to be rinsed, more particularly formulations of the conditioner type.

By way of examples of such additives, mention may be made of cationic conditioners, styling agents, volumizing agents or fixing agents for the hair, repairing, nourishing or moisturizing agents, water-soluble monovalent mineral salts, dyes, fragrances, vitamins.

More particularly, they may comprise at least one cationic polymer that is soluble in the medium of the formulation.

By way of example, mention may be made of polymers of the polyquaternium family, and cationic derivatives of polysaccharides, such as cationic derivatives of guar or of cellulose. By way of illustration, polymers that may be suitable include cationic polymers functionalized with hydrophobic groups such as C₁-C₁₄ alkyl chains, attached to the main polymeric chain by means of ether linkages and optionally having a hydroxyl group, the cationic group being more particularly a quaternary ammonium group carrying three radicals, which may or may not be identical, chosen from hydrogen and an alkyl radical comprising 1 to 22 carbon atoms, more particularly 1 to 14, advantageously 1 to 3 carbon atoms. The counterion is a halogen, preferable chlorine.

In the case of the modified cationic celluloses, which may or may not be hydrophobic, the cationic group is a quaternary ammonium group carrying three radicals, which may or may not be identical, chosen from hydrogen and an alkyl radical comprising 1 to 10 carbon atoms, more particularly 1 to 6, advantageously 1 to 3 carbon atoms. The counterion is a halogen, preferably chlorine.

Among the guar derivatives and cellulose derivatives, mention may be made of guar hydroxypropyl trimonium chloride (Jaguar C13S, C14S, or C17 sold by the company Rhodia Chimie) or hydroxypropyl guar hydroxypropyl trimonium chloride (Jaguar C162 sold by Rhodia), or cellulose poly(oxyethanediyl-1,2) 2-hydroxy-3-(trimethylammonium)propyl ether, chloride or polyquaternium-10, such as the polymer JR400 sold by the company Union Carbide.

Synthetic polymers, and more particularly homopolymers such as polymethacrylamidopropyl trimonium chloride (Polycare 133 sold by the company Rhodia Chimie) may similarly be suitable.

The formulation that is the subject of the invention may also contain fixative resins which, when they are present, are more particularly present in an amount of 0.01 to 10%, preferably between 0.5 and 5% by weight of the formulation.

For example, the fixative resins may be chosen from polyvinylpyrrolidone, copolymers of polyvinylpyrrolidone and of methyl methacrylate, copolymers of polyvinylpyrrolidone and of vinyl acetate, poly(ethylene glycol terephthalate)/poly(ethylene glycol) copolymers, and mixtures thereof.

The formulation may also contain polymeric derivatives that exercise a protective function, the content of which, if they are present, is in general between 0.01 and 10% by weight of the formulation.

These agents may in particular be chosen from nonionic cellulose derivatives such as cellulose hydroxy ethers, methylcellulose, ethylcellulose, hydroxypropylmethylcellulose, hydroxybutylmethylcellulose; poly(vinyl ester)s grafted onto polyalkylene backbones, such as poly(vinyl acetate)s grafted onto polyoxyethylene backbones (EP-A-219 048); poly(vinyl alcohol)s.

It is also possible to advantageously add to the formulation metal-sequestering agents, more particularly those that sequester the calcium, for instance citrate ions.

It is also possible to incorporate into the cosmetic compositions that are the subject of the invention humectants, which include, inter alia, glycerol, sorbitol, urea, collagen, gelatin, aloe vera, hyaluronic acid or water-soluble volatile solvents such as ethanol or propene glycol, the contents of which may go up to 60% by weight of the composition.

It is possible to add to these compounds, in combination, mineral powders or particles such as calcium carbonate, sodium bicarbonate, calcium dihydrogen phosphate, mineral oxides in the form of powder or in colloidal form (particles of size less than or of the order of a micrometer, sometimes of a few tens of nanometers), such as titanium dioxide, silica, aluminum salts generally used as antiperspirants, kaolin, talc, clays, and derivatives thereof, etc.

Preserving agents such as methyl, ethyl, propyl and butyl esters of p-hydroxybenzoic acid, sodium benzoate, Germaben® or any chemical agent that prevents the proliferation of bacteria or of molds and that is conventionally used in cosmetic compositions, can also be introduced into the formulation according to the invention, generally in an amount of 0.01 to 3% by weight, adjusted to prevent proliferation of bacteria, molds or yeasts.

As an alternative to these chemical agents, agents that modify water activity and strongly increase osmotic pressure, such as carbohydrates or salts, can sometimes be used.

In order to protect the skin and/or the hair against attacks from sunlight and from UV-rays mineral particles such as zinc oxide, titanium dioxide or cerium oxides, in the form of powder or of colloidal particles, alone or as a mixture, can be added to these formulations. These powders can optionally be surface-treated so as to increase the effectiveness of their anti-UV action or so as to facilitate their incorporation into the cosmetic formulations or so as to inhibit the surface photoreactivity.

One or more fragrances, dyeing agents and/or opacifiers, such as pigments, can be added to these ingredients, if necessary, and for the purpose of increasing the comfort when the composition is used by the consumer.

Although this is not obligatory, the composition can also contain viscosity-modifying or gelling polymers so as to adjust the texture of the composition, such as crosslinked polyacrylates (Carbopol sold by Goodrich), noncationic derivatives of cellulose such as hydroxypropylcellulose or carboxymethylcellulose, guars and their nonionic derivatives, used alone or in combination.

The formulation can similarly contain polymeric dispersing agents in an amount of the order of 0.1-7% by weight, in order to control the calcium and magnesium hardness, agents such as:
water-soluble salts of polycarboxylic acids having a molecular mass by weight of the order of 2000-100 000 g/mol, obtained by polymerization or copolymerization of ethylenically unsaturated carboxylic acids such as acrylic acid, maleic acid or maleic anhydride, fumaric acid, itaconic acid, aconitic acid, mesaconic acid, citraconic acid or methylenemalonic acid, and most particularly polyacrylates having a molecular mass by weight of the order of 2000 to 10 000 g/mol (U.S. Pat. No. 3,308,067), and copolymers of acrylic acid and of maleic anhydride having a molecular mass by weight of the order of 5000 to 75 000 g/mol (EP-A-66 915);
poly(ethylene glycol)s having a molecular mass by weight of the order of 1000 to 50 000 g/mol.

A second subject of the invention consists of a method of treating keratinous material, such as the skin and the hair, by bringing said material into contact with the formulation described above and then rinsing it with an aqueous rinsing medium.

More particularly, the amount of formulation used, expressed as solids content, is from 0.001 to 10 g/l, preferably from 0.05 to 2 g/l during the rinsing process.

In addition, the rinsing medium advantageously has a pH of 5.5 to 8.

It is specified that the formulation can be applied to a dry or wet surface.

In accordance with a very advantageous embodiment of the invention, the active material present in the formulation contains, in encapsulated, dispersed or solubilized form, at least one liquid or solid hydrophobic active compound that is different from the active material. According to this embodiment, the method is intended to provide the keratinous material with the additional benefits intrinsic to said hydrophobic organic active compound.

The presence of the carrier agent makes it possible to increase the deposition of the organic hydrophobic active compound, encapsulated, dispersed and/or alternatively solubilized in the active material, onto the surface of the hair or of the skin. This results in an increased retention of this active compound on the treated surface, and in this surface being provided more effectively with the intrinsic properties of the active compound. This is particularly notable when the organic active compound is a fragrance.

Another subject of the invention consists of a method intended to improve the volumizing properties and/or the properties that help styling and/or the properties consisting of a fixing effect for keratinous fibers, consisting in bringing said fibers into contact with the formulation according to the invention, and then in rinsing said fibers with an aqueous rinsing medium.

Preferably, the active material comprises a hydrophobic organic active compound encapsulated, solubilized or alternatively dispersed in the active material.

The invention similarly relates to a method for improving the depositing of an active material onto keratinous material, during which a formulation is applied to said material and then a rinsing process is carried out with an aqueous rinsing medium;

said formulation comprising at least one active material and, optionally, at least one salt that is soluble in the formulation, and being in the form of a stable dispersion, the pH of which is between 3 and 5.5, preferably between 4.5 and 5.5,

the active material containing, optionally in an encapsulated, dispersed or solubilized form, at least one hydrophobic organic active compound that is different from the active material; said active material, which may or may not be in a liquid form, having, in the medium of the formulation, an overall cationic or zero charge, being insoluble in the medium of the formulation, being stabilized in the medium of the formulation by means of a cationic surfactant, and remaining insoluble in the rinsing medium or being capable of swelling in the rinsing medium;

by addition of at least one carrier agent consisting of at least one organic polymer that is soluble or dispersible in the medium of the formulation and in the rinsing medium, having, in the medium of the formulation, an overall ionic charge that is zero or cationic and being capable of developing, at the pH of the rinsing process in the rinsing medium, a sufficient number of anionic charges to destabilize the active material in the rinsing medium.

It is specified that the amount of formulation used, expressed as solids content, is usually from 0.001 to 10 g/l, preferably from 0.05 to 2 g/l during the rinsing process.

In addition, the rinsing medium advantageously has a pH of 5.5 to 8.

Finally, a subject of the invention is the use, in a formulation intended to be applied to keratinous material, for example the skin and/or the hair, and to be rinsed with an aqueous rinsing medium, said formulation being in the form of a stable dispersion, the pH of which is between 3 and 5.5, preferably between 4.5 and 5.5, and comprising:

at least one active material, which may or may not be in a liquid form, that has, in the medium of the formulation, an overall cationic or zero charge, that is insoluble in the medium of the formulation, that is stabilized in the medium of the formulation by means of a cationic surfactant, and that remains insoluble in the rinsing medium or is capable of swelling in the rinsing medium;

optionally, at least one salt that is soluble in the formulation;

of at least one carrier agent consisting of at least one organic polymer that is soluble or dispersible in the medium of the formulation and in the rinsing medium, that has, in the medium of the formulation, an overall ionic charge that is zero or cationic and that is capable of developing, at the pH of the rinsing process in the rinsing medium, a sufficient number of anionic charges to destabilize the active material in the rinsing medium,

as an agent capable of bringing said active material to the surface of the keratinous fibers during the rinsing process.

The amount of formulation used, expressed as solids content, is advantageously from 0.001 to 10 g/l, preferably from 0.05 to 2 g/l during the rinsing process.

In addition, the rinsing medium advantageously has a pH of 5.5 to 8.

The following examples are given by way of illustration.

### EXAMPLE 1

Formulation I1.

An aqueous (latex) dispersion of a poly(butyl acrylate) (active material A) having a molar mass by weight of 500 000 g/mol and a particle size of 35 nm, obtained by emulsion polymerization of butyl acrylate in the presence of 10 parts by weight of cetyltrimethylammonium bromide (surfactant TAC) per 100 parts by weight of butyl acrylate is used.

The dispersion has a solids content of 28% by weight.

The pH of this dispersion is adjusted to 4.0 with a 1 N hydrochloric acid solution.

The dispersion is slightly opalescent.

20 ml of the dispersion are poured, with stirring, into 1 ml of water, the pH of which has been adjusted to 4. A mixture is obtained at pH=4, the appearance of which is not significantly different from that of the original dispersion. This mixture is stable for several days, it does not form any precipitate and does not change in terms of turbidity over time.

Formulation II1.

An aqueous (latex) dispersion of a poly(butyl acrylate) (active material) having a particle size of 35 nm, obtained by emulsion polymerization of butyl acrylate in the presence of 10 parts by weight of cetyltrimethylammonium bromide (surfactant) per 100 parts by weight of butyl acrylate is used.

The dispersion has a solids content of 28% by weight.

The pH of this dispersion is adjusted to 4.0 with a 1 N hydrochloric acid solution. The dispersion is slightly opalescent.

Furthermore, an aqueous solution containing 2.2% by weight of a copolymer (carrier agent) of acrylic acid and of DADMAC (molar mass of 100 000 g/mol), with a 1/1 molar ratio, is prepared, the pH of which solution was adjusted to 4.0 with a 10% by weight hydrochloric acid solution.

20 ml of the dispersion of active material is poured into 1 ml of the solution of carrier agent copolymer with mechanical stirring. A mixture is obtained at pH=4, the appearance of which is not significantly different from that of the original dispersion. This mixture is stable for several days, it does not form any precipitate and does not change in terms of turbidity over time.

Diluted Formulations I1(a) and II1(a) Obtained by Dilution of the Formulations I1 and II1 in Water at pH=4

Two beakers are prepared, each containing 200 ml of water, the pH of which has been adjusted to 4 with hydrochloric acid.

0.1 ml of the formulation I1 is added, with mechanical stirring to one beaker and 0.1 ml of the formulation II1 is added, with mechanical stirring, to the other, and the turbidity of the mixture is followed over time. The change in turbidity remains slight, and no particle formation is seen over time. The results are given in table 1.

The turbidity measurements were carried out using a Metrohm turbidimetry cell coupled to a Metrohm photometer; the turbidity value is obtained by comparing the light intensity transmitted in the cell in air, and then in the medium studied.

TABLE 1

| | Turbidity | Turbidity |
|---|---|---|
| | (arbitrary units) | (arbitrary units) |
| Time (min) | formulation I1(a) | formulation II1(a) |
| 1 | 0.02 | 0.03 |
| 2 | 0.023 | 0.033 |
| 3 | 0.025 | 0.035 |
| 4 | 0.027 | 0.037 |
| 5 | 0.029 | 0.039 |
| 7 | 0.032 | 0.043 |
| 10 | 0.036 | 0.047 |

Diluted Formulations I1(b) and II1(b) Obtained by Dilution of the Formulations I1 and II1 in water at pH=7.2

Two beakers are prepared, each containing 200 ml of water at its natural pH of 7.2.

0.1 ml of the formulation I1 is added, with mechanical stirring, to one beaker and 0.1 ml of the formulation II1 is added, with mechanical stirring, to the other, and the turbidity of the mixture is followed over time. The results are given in table 2.

It is noted that the turbidity of the solution containing the copolymer of acrylic acid and of DADMAC greatly increases over time.

TABLE 2

| | Turbidity | Turbidity |
|---|---|---|
| | (arbitrary units) | (arbitrary units) |
| Time (min) | formulation I1(b) | formulation II1(b) |
| 1 | 0.024 | 0.025 |
| 2 | 0.036 | 0.067 |
| 3 | 0.049 | 0.149 |
| 4 | 0.059 | 0.28 |
| 5 | 0.068 | 0.472 |
| 7 | 0.087 | 0.633 |
| 10 | 0.118 | 0.725 |

The formulation II1(b) makes it possible to increase the amount of latex deposited onto the hair at the time of dilution.

Influence of the (Carrier Agent)/(Active Material) Weight Ratio:

An aqueous (latex) dispersion of a poly(butyl acrylate) (active material) having a particle size of 35 nm, obtained by emulsion polymerization of butyl acrylate in the presence of 10 parts by weight of cetyltrimethylammonium bromide (surfactant) per 100 parts by weight of butyl acrylate is used.

The dispersion has a solids content of 28% by weight.

The pH of this dispersion is adjusted to 4.0 with a 1 N hydrochloric acid solution. The dispersion is slightly opalescent.

Furthermore, an aqueous solution containing 22% by weight of a copolymer (carrier agent) of acrylic acid and of DADMAC (of molar mass 100 000 g/mol), with a 1/1 molar ratio, is prepared, the pH of which solution was adjusted to 4.0 with a 10% by weight hydrochloric acid solution.

20 ml of the dispersion of active material are poured into, respectively, 4 ml (formulation 1), 0.1 ml (formulation 2), 0.05 ml (formulation 3) and 0.025 ml (formulation 4) of the solution of carrier agent copolymer, with mechanical stirring.

The volumes of formulations 2, 3 and 4 are made up with, respectively, 3.9 ml, 3.95 ml and 3.975 ml of water, in order to keep the active material concentration constant.

Four formulations of pH=4 are obtained, the appearance of which is not significantly different from that of the original dispersion, for which the (carrier agent)/(active material) weight ratios are, respectively, 14.8/100; 0.37/100; 0.18/100 and 0.09/100.

Four beakers are prepared, each containing 200 ml of water at its natural pH of 7.2.

0.1 ml of formulation is added to each beaker, with mechanical stirring, and the turbidity of the mixture is followed over time.

The turbidity measurements were carried out using a Metrohm turbidimetry cell coupled to a Metrohm photometer; the turbidity value is obtained by comparing the light intensity transmitted in the cell in air, and then in the medium studied.

The turbidity measurement results are given in table 3 below:

TABLE 3

Turbidity (arbitrary units)

| Time | (C)/(A) | (C)/(A) | (C)/(A) | (C)/(A) |
|---|---|---|---|---|
| (min) | 14.8/10 | 0.37/100 | 0.18/100 | 0.09/100 |
| 1 | 0.02 | 0.03 | 0.03 | 0.03 |
| 2 | 0.015 | 0.07 | 0.035 | 0.04 |
| 5 | 0.05 | 0.48 | 0.09 | 0.075 |
| 7 | 0.04 | 0.64 | 0.15 | 0.09 |
| 10 | 0.03 | 0.72 | 0.22 | 0.12 |

These results show that the (carrier agent)/(active material) ratio has an influence on the ability of the carrier agent to cause flocculation of the active material.

### EXAMPLE 2

30% of a sunflower oil of the Lubrirob® TOD18.80 type (from Rhodia-Novance) is emulsified in water in a microfluidizer (4 bar, 3 cycles) under hot conditions (50° C.) in the presence of 3% by weight of cationic surfactants (of the cetyltrimethylammonium bromide type).

An emulsion (E) having a solids content of 30% by weight of active material is obtained, the size of which emulsion, measured by laser diffraction (Horiba granulometer), is 250 nm. This size is a mean size by mass of the size distribution of the emulsion.

This emulsion (E) is used to prepare various formulations.

Formulation I2.

The pH of the emulsion (E) is adjusted to 4.0 with a 1 N hydrochloric acid solution. The dispersion obtained is milky.

20 ml of the dispersion are poured, with mechanical stirring, into 1 ml of water, the pH of which has been adjusted to 4. A mixture at pH=4 is obtained, the appearance of which is not significantly different from that of the original dispersion. This mixture is stable for several days, it does not form any precipitate and does not change in terms of turbidity over time.

Formulation II2.

The pH of the emulsion (E) is adjusted to 4.0 with a 1 N hydrochloric acid solution. The dispersion obtained is milky.

Furthermore, an aqueous solution containing 2.2% by weight of a (carrier agent) copolymer of acrylic acid and of DADMAC (of molar mass 100 000 g/mol), with a 1/1 molar ratio, is prepared, the pH of which solution was adjusted to 4.0 with a 10% by weight hydrochloric acid solution.

20 ml of the emulsion (E) are poured into 1 ml of the solution of carrier agent copolymer, with mechanical stirring. A mixture is obtained at pH=4, the appearance of which is not significantly different from that of the original dispersion. This mixture is stable for several days, it does not form any precipitate and does not change in terms of turbidity over time.

Diluted Formulations I2(a) and II2(a) Obtained by Dilution of the Formulations I2 and II2 in Water at pH=4

Water, the pH of which has been adjusted to 4 (approximately 100 ml), is introduced into a Horiba granulometer tank.

0.1 ml of the formulation 12 is added thereto, with mechanical stirring, and the change in the size of the emulsion is followed over time.

The same process is repeated with 0.1 ml of the formulation II2.

The results are given in table 4.

TABLE 4

| | Emulsion size in nm | Emulsion size in nm |
|---|---|---|
| Time (min) | formulation I2(a) | formulation II2(a) |
| 1 | 525 | 525 |
| 5 | 525 | 525 |
| 10 | 525 | 525 |

This size does not change over time.

Diluted Formulations I2(b) and II2(b) Obtained by Dilution of the Formulations I2 and II2 in Water at pH=7.2

Water at its natural pH of 7.2 (approximately 100 ml) is introduced into a granulometer tank. 0.1 ml of the formulation 12 is added thereto, with mechanical stirring, and the change in size of the emulsion is followed over time.

The same process is repeated with 0.1 ml of the formulation II2.

The results are given in table 5.

TABLE 5

| | Emulsion size in nm | Emulsion size in nm |
|---|---|---|
| Time (min) | formulation I2(b) | formulation II2(b) |
| 1 | 525 | 750 |
| 5 | 525 | 1230 |
| 10 | 525 | 1800 |

The formulation II2(b) makes it possible to increase the amount of latex deposited onto the hair at the time of dilution.

Influence of the (Carrier Agent)/(Active Material) Weight Ratio:

The pH of the emulsion (E) containing 30% by weight of active material is adjusted to 4.0 with a 1 N hydrochloric acid solution. The dispersion obtained is milky.

Furthermore, an aqueous solution containing 11% by weight of a (carrier agent) copolymer of acrylic acid and of DADMAC (of molar mass 100 000 g/mol), with a 1/1 molar ratio, is prepared, the pH of which solution was adjusted to 4.0 with a 10% by weight hydrochloric acid solution.

20 ml of the emulsion (E) are poured into, respectively, 4 ml (formulation 1), 0.1 ml (formulation 2) and 0.05 ml (formulation 3) of the solution of carrier agent copolymer, with mechanical stirring.

The volumes of formulations 2 and 3 are made up with, respectively, 3.9 ml and 3.95 ml of water, in order to keep the active material concentration constant.

Three formulations of pH=4 are obtained, the appearance of which is not significantly different from that of the original dispersion, for which the (carrier agent)/(active material) weight ratios are, respectively, 7.2/100; 0.18/100 and 0.09/100.

Three beakers are prepared, each containing 200 ml of water at its natural pH of 7.2.

0.1 ml of formulation is added to each beaker, with mechanical stirring, and the change in appearance of the dispersions is followed.

| | (C)/(A) (*) | (C)/(A) (*) | (C)/(A) (*) |
|---|---|---|---|
| | 7.2/100 | 0.18/100 | 0.09/100 |
| Appearance | Stable milky | Flocculation | Flocculation |

(*) (C)/(A): carrier agent/active material weight ratio

These results show that the (C)/(A) ratio has an influence on the ability of the carrier agent to cause flocculation of the active material.

1-48. (canceled)

49. A formulation intended to be applied to keratinous material and to be rinsed with an aqueous rinsing medium in the form of a stable dispersion, the pH of which is between 3 and 5.5, and comprising at least one active material, a carrier agent consisting of at least one organic polymer capable of bringing said active material to the surface of the keratinous material during the rinsing process and, optionally, at least one salt that is soluble in the formulation; the nature of the active material and of the carrier agent being such that:
the active material:
is optionally in a liquid form,
has, in the medium of the formulation, an overall cationic or zero charge,
is insoluble in the medium of the formulation,
is stabilized in the medium of the formulation by means of a cationic and/or nonionic surfactant, and
remains insoluble in the rinsing medium or is capable of swelling in the rinsing medium;
the carrier agent:
is soluble or dispersible in the medium of the formulation and in the rinsing medium,
has, in the medium of the formulation, an overall ionic charge that is zero or cationic,
is capable of developing, at the pH of the rinsing process in the rinsing medium, a sufficient number of anionic charges to destabilize the active material in the rinsing medium.

50. The formulation as claimed in claim 49, wherein the rinsing medium has a pH of 5.5 to 8.

51. The formulation as claimed in claim 49, wherein the active material is in the form of solid particles dispersed in the medium of the formulation and is:
a) a nonionic polymer derived from at least one nonionic hydrophobic monomer,
b) a polymer derived from at least one nonionic hydrophobic monomer and from at least one monomer that is cationic or potentially cationic in the medium of the formulation and, optionally, from at least one monomer that is neutral in the medium of the formulation and potentially anionic in the rinsing medium, or
c) a polymer derived from at least one nonionic hydrophobic monomer and from at least one monomer that is neutral in the medium of the formulation and potentially anionic in the rinsing medium.

52. The formulation as claimed in claim 51, wherein the monomer composition from which said polymer derives contains:
at least one uncharged or non-ionizable hydrophilic monomer, preferably in an amount that does not exceed 50% of the total mass of monomers,
and/or at least one zwitterionic monomer, optionally in an amount that does not exceed 30% of the total mass of monomers,
and/or at least one crosslinking monomer, optionally in an amount that does not exceed 10% of the total mass of monomers.

53. The formulation as claimed in claim 51, wherein the polymer b) contains an anionic monomer, the first pKa of which is less than 6, optionally 3, this being in a sufficiently small amount so that said polymer b) has, in the medium of the formulation, an overall cationic charge.

54. The formulation as claimed in claim 51, wherein, when the active material is an ionic or ionizable polymer, the choice and the relative amounts of monomers from which the copolymer derives are such that the active material:
is insoluble in the medium of the formulation;
has, in the formulation, an overall cationic or zero charge; and
remains insoluble in the rinsing medium or is not capable of swelling by more than 8 times, preferably not by more than 4 times, its volume in the rinsing medium.

55. The formulation as claimed in claim 49, wherein the active material is in the form of particles of polymer, the mean diameter of which is between 10 nm and 10 μm.

56. The formulation as claimed in claim 49, wherein the active material is a polymer that derives from monomers that are α-β monoethylenically unsaturated or diethylenically unsaturated in the case of the crosslinking monomers.

57. The formulation as claimed in claim 49, wherein the active material is a polymer that derives from monomers, the choice and the relative amounts of which are such that said polymer has a glass transition temperature Tg of −80° C. to +150° C., optionally of −80° C. to +40° C.

58. The formulation as claimed in claim 49, wherein the active material is a polymer that is insoluble in the medium of the formulation and in the rinsing medium, chosen from the polymers derived from at least one nonionic hydrophobic monomer and the polymers derived from at least one nonionic hydrophobic monomer and from 0.1 to 20% of their weight of at least one monomer that is potentially cationic in the medium of the formulation.

59. The formulation as claimed in claim 49, wherein the active material is a polymer capable of swelling in the rinsing medium, chosen from the polymers derived from at least one nonionic hydrophobic monomer and from 10 to 50% of its weight of at least one monomer that is potentially anionic in the rinsing medium.

60. The formulation as claimed in claim 49, wherein the active material in the form of particles of polymer contains, encapsulated in its particles, at least one liquid or solid hydrophobic organic active compound that is different from the active material.

61. The formulation as claimed in claim 49, wherein the cationic charges, brought about by the possible cationic or potentially cationic units of the active material in the form of polymer and possibly by the cationic surfactant(s), at the surface of the active material dispersed in the medium of the formulation, are such that the zeta potential of the active material dispersed in the medium of the formulation is from 0 to +50 mV.

62. The formulation as claimed in claim 49, wherein the active material is selected from the group consisting of mineral oils; organic oils; fats; waxes; silicone oils; resins; gums; aromas; essential oils; fragrances; antimicrobial agents; liposoluble vitamins; phospholipids; bactericides; and UV-absorbing agents.

63. The formulation as claimed in claim 49, wherein the active material further comprises, solubilized or dispersed, at least one liquid or solid hydrophobic organic active compound that is different from the active material.

64. The formulation as claimed in claim 63, wherein the weight amount of cationic and/or nonionic surfactant in the formulation is less than or equal to 25% by weight of the formula, optionally less than or equal to 5%.

65. The formulation as claimed in claim 49, wherein the medium of the formulation is an aqueous or aqueous-alcoholic medium.

66. The formulation as claimed in claim 65, wherein the alcohols present in the aqueous-alcoholic medium represent up to 70% of the volume of the medium of the formulation.

67. The formulation as claimed in claim 49, wherein the carrier agent is a polymer that is soluble or dispersible in an aqueous or aqueous-alcoholic medium having a pH of between 3 and 8, comprising at least one unit that is neutral in the medium of the formulation and potentially anionic in the rinsing medium.

68. The formulation as claimed in claim 67, wherein the carrier agent comprises at least one unit that is cationic or potentially cationic in the medium of the formulation and/or at least one hydrophilic or hydrophobic, nonionic unit.

69. The formulation as claimed in claim 49, wherein the relative amounts of the various units of the polymer constituting the carrier agent are such that, in the medium of the formulation, the overall charge of the carrier agent is zero or cationic.

70. The formulation as claimed in claim 49, wherein the relative amounts of carrier agent, optionally of cationic surfactant and of active material are such that, during the rinsing process, the number of anionic charges developed in the rinsing medium by the carrier agent is sufficient to destabilize the active material in the rinsing medium.

71. The formulation as claimed in claim 70, wherein the number of anionic charges developed in the rinsing medium by the carrier agent so as to destabilize the active material is at least 1% relative to the number of surface cationic charges of the active material in the rinsing medium, and at most 200% relative to the number of surface cationic charges of the active material in the rinsing medium.

72. The formulation as claimed in claim 49, wherein the carrier agent is a polymer chosen from polymers derived from ethylenically unsaturated monomers, natural polysaccharides that are potentially anionic, and substituted or modified polysaccharides that are potentially anionic or amphoteric.

73. The formulation as claimed in claim 49, wherein the carrier agent is a polymer derived:
from at least one α-β monoethylenically unsaturated monomer that is neutral in the medium of the formulation and potentially anionic in the rinsing medium, and,
optionally, from at least one α-β monoethylenically unsaturated monomer that is cationic or potentially cationic in the medium of the formulation, and
optionally, from at least one hydrophilic or hydrophobic, preferably hydrophilic, nonionic α-β monoethylenically unsaturated monomer.

74. The formulation as claimed in claim 49, wherein the carrier agent is a random, block or grafted polymer derived:
from at least one α-β monoethylenically unsaturated hydrophilic monomer that is neutral in the medium of the formulation and potentially anionic in the rinsing medium, and
from at least one α-β monoethylenically unsaturated hydrophilic monomer that is cationic or potentially cationic in the medium of the formulation, and,
optionally, from at least one hydrophilic or hydrophobic, nonionic α-β monoethylenically unsaturated monomer.

75. The formulation as claimed in claim 49, wherein the carrier agent derives from one or more α-β monoethylenically unsaturated monomers and has a mean molar mass by weight of greater than 5000 g/mol.

76. The formulation as claimed in claim 49, wherein the carrier agent is selected from the group consisting of:
polyacrylic or polymethacrylic acids, alkali metal polyacrylates or polymethacrylates, optionally having a mean molar mass by weight of 100 000 to 1 000 000 g/mol;
acrylic acid/DADMAC polymers, having a molar ratio of 50/50 to 30/70, optionally having a mean molar mass by weight of 70 000 to 350 000 g/mol;
acrylic acid/MAPTAC polymers, having a molar ratio of 60/40 to 30/70, optionally having a mean molar mass by weight of 90 000 to 300 000 g/mol;
acrylic acid/MAPTAC/linear C₄-C₁₈ alkyl methacrylate polymers comprising from 0.005 to 10% by mass of alkyl methacrylate, with an acrylic acid/MAPTAC molar ratio ranging from 60/40 to 30/70, and optionally having a mean molar mass by weight of 50 000 to 250 000 g/mol, and
acrylic acid/dimethylaminoethyl methacrylate (DMAEMA) polymers, having a molar ratio of 60/40 to 30/70, optionally having a a mean molar mass by weight of 50 000 to 300 000 g/mol.

77. The formulation as claimed in claim 49, wherein the carrier agent is a potentially anionic natural polysaccharide formed of nonionic monosaccharide units and of monosaccharide units that are neutral in the medium of the formulation and potentially anionic in the rinsing medium, these units being identical or different.

78. The formulation as claimed in claim 77, wherein the potentially anionic natural polysaccharide is a branched polysaccharide formed:
of a main chain comprising anhydrohexose units that may be similar or different, and
of branches comprising at least one anhydropentose and/or anhydrohexose unit that is neutral in the medium of the formulation and optionally potentially anionic in the rinsing medium.

79. The formulation as claimed in claim 77, wherein said potentially anionic natural polysaccharide is a xanthan gum, a succinoglycan, a rhamsan, a gellan gum or a welan gum.

80. The formulation as claimed in claim 77, wherein said potentially anionic natural polysaccharide has a mean molar mass by weight of 2000 to 5 000 000 g/mol.

81. The formulation as claimed in claim 49, wherein the carrier agent is a substituted or modified polysaccharide, the natural backbone of which is formed of nonionic monosaccharide units and/or of monosaccharide units that are neutral in the medium of the formulation and potentially anionic in the rinsing medium, said monosaccharide units being identical or different, and being substituted or modified:
with one or more group(s) which carries or carry at least one charge that is neutral in the medium of the formulation and potentially anionic in the rinsing medium, and
optionally, with one or more group(s) which carry or carries at least one charge that is cationic or potentially cationic in the medium of the formulation, the degree of substitution or of modification of the monosaccharide units with the entirety of the groups which carry charges that are potentially anionic and of optional groups which carry cationic charges being such that said substituted or modified polysaccharide is soluble or dispersible in an aqueous or aqueous-alcoholic medium and has an overall zero or cationic charge in the medium of the formulation.

82. The formulation as claimed in claim 81, wherein said substituted or modified polysaccharide contains at least one nonionic modifying group or substituent group.

83. The formulation as claimed in claim 81, wherein said substituted or modified polysaccharide is a branched substituted or modified polysaccharide, the natural backbone of which is formed:
from a main chain comprising similar or different anhydrohexose units, and from branches comprising at least one anhydropentose and/or anhydrohexose unit that is neutral in the medium of the formulation and optionally potentially anionic in the rinsing medium,
the anhydrohexose and/or anhydropentose units of said polysaccharide being substituted or modified with one or more groups which carry at least one charge that is neutral in the medium of the formulation and potentially anionic in the rinsing medium, and optionally at least one charge that is cationic or potentially cationic in the rinsing medium,
the degree of substitution or of modification DSi of the anhydrohexose and/or anhydropentose units with the entirety of said groups which carry charges that are ionic or potentially ionic ranging from 0.01 to less than 3,
with a ratio of the number of charges that are potentially anionic in the rinsing medium to the number of charges that are cationic or potentially cationic in the medium of the formulation ranging from 100/0 to 30/70.

84. The formulation as claimed in claim 81, wherein said substituted or modified polysaccharide has a mean molar mass by weight of 2000 to 5 000 000 g/mol, optionally of 10 000 to 5 000 000 g/mol.

85. The formulation as claimed in claim 81, wherein the natural backbone of said substituted or modified polysaccharide is a galactomannan.

86. The formulation as claimed in claim 81, wherein the natural backbone of said substituted or modified polysaccharide iscarboxymethylgalactomannans, carboxymethylguars, carboxymethylhydroxypropylgalactomannans, carboxymethylhydroxypropylguars, carboxymethylhydroxypropyltrimethylammonium chloride galactomannans, carboxymethylhydroxypropyltrimethylammonium chloride guars, carboxymethylhydroxypropyl-hydroxypropyltrimethylammonium chloride galactomannans, or carboxymethylhydroxpropyl-hydroxypropyltrimethylammonium chloride guars.

87. The formulation as claimed in claim 49, wherein the amount of carrier agent present in said formulation is between 0.001 and 50 parts by weight, and 2 parts by weight per 100 parts by weight of active material.

88. The formulation as claimed in claim 49, having at least one soluble salt chosen from chlorides, bromides, iodides, nitrates, sulfates and sulfonates of an alkali metal, or of ammonium.

89. The formulation as claimed in claim 49, being in the form of an aqueous or aqueous-alcoholic dispersion comprising per 100 parts of its weight:
from 0.01 to 50, parts by dry weight of active material,
from 0.01 to 35, parts by dry weight of cationic surfactant,
from 0.001 to 5, parts by dry weight of carrier agent, and
at most 2 parts by weight of soluble salt.

90. The formulation as claimed in claim 49, further comprising one or more usual constituents selected from the group consisting of cationic conditioners, styling agents, volumizing agents for the hair, fixing agents for the hair, repairing, nourishing, moisturizing agents, water-soluble monovalent mineral salts, dyes, fragrances, and vitamins.

91. A method of treating keratinous material comprising the steps of:
a) bringing said material into contact with the formulation as claimed in claim 49, and, then,
b) rinsing it with an aqueous rinsing medium.

92. The method as claimed in claim 91, wherein the active material contains, in encapsulated, dispersed or solubilized form, at least one liquid or solid hydrophobic active compound that is different from the active material.

93. A method intended to improve the volumizing properties and/or the properties that help styling and/or the properties consisting of a fixing effect for keratinous fibers, comprising the steps of:
a) bringing said fibers into contact with the formulation as claimed in claim 49, and, then,
b) rinsing said fibers with an aqueous rinsing medium.

94. A method for improving the depositing of an active material onto keratinous material, comprising the steps of:
a) applying a formulation to said material and, then,
b) rinsing said material with an aqueous rinsing medium;
said formulation comprising at least one active material and, optionally, at least one salt that is soluble in the formulation, and being in the form of a stable dispersion, the pH of which is between 3 and 5.5,
the active material containing, optionally in an encapsulated, dispersed or solubilized form, at least one hydrophobic organic active compound that is different from the active material; said active material, optionally being in a liquid form, and having, in the medium of the formulation, an overall cationic or zero charge, being insoluble in the medium of the formulation, being stabilized in the medium of the formulation by means of a cationic surfactant, and remaining insoluble in the rinsing medium or being capable of swelling in the rinsing medium;
by addition of at least one carrier agent consisting of at least one organic polymer that is soluble or dispersible in the medium of the formulation and in the rinsing medium, having, in the medium of the formulation, an overall ionic charge that is zero or cationic and being capable of developing, at the pH of the rinsing process in the rinsing medium, a sufficient number of anionic charges to destabilize the active material in the rinsing medium.

95. The method as claimed in claim 91, wherein in step b) the formulation is being used in an amount, expressed as solids content, of from 0.001 to 10 g/l.
